(19) United States
(12) Patent Application Publication
(10) Pub. No.: US 2015/0065466 A1
(43) Pub. Date: Mar. 5, 2015

(54) NOVEL DXR INHIBITORS FOR ANTIMICROBIAL THERAPY

(71) Applicant: Baylor College of Medicine, Houston, TX (US)

(72) Inventor: Yongcheng Song, Pearland, TX (US)

(21) Appl. No.: 14/390,736

(22) PCT Filed: Apr. 18, 2013
(86) PCT No.: PCT/US13/37119

§ 371 Date: Oct. 3, 2014

Related U.S. Application Data
(60) Provisional application No. 61/636,036, filed on Apr. 20, 2012.

(51) Int. Cl. C07F 9/58; C07F 9/6521; A61K 45/06; C07F 9/6506; A61K 31/675; A61K 31/662; C07F 9/6512; C07F 9/38

(52) U.S. Cl. CPC C07F 9/582; C07F 9/65122; C07F 9/65212; C07F 9/3808; C07F 9/65062; A61K 31/675; A61K 31/662; A61K 45/06
USPC 514/84; 546/22; 544/243; 544/214; 562/11; 548/119; 514/89; 514/86; 514/119; 514/94

(57) ABSTRACT

The present invention generally concerns particular methods and compositions for antimicrobial therapy. In particular embodiments, the compositions target DXR. In some cases, the antimicrobial agent comprises an electron-deficient hydrophobic group that has interacts with Trp211 of DXR. In specific embodiments, the compound contains electron-deficient heterocyclic rings that specifically interact with the electron-rich indole ring of Trp211. In certain aspects, the compositions comprise a phosphate group, a pyridine group, and a hydroxymate group.

This application claims priority to U.S. Provisional Patent Application Ser. No. 61/636,036, filed Apr. 20, 2012, which is incorporated by reference herein in its entirety.

### STATEMENT REGARDING FEDERALLY SPONSORED RESEARCH OR DEVELOPMENT

This invention was made with government support under R21AI088123 awarded by National Institute for Allergy and Infectious Diseases (NIAID). The government has certain rights in the invention.

### TECHNICAL FIELD

The present invention generally concerns at least the fields of cell biology, molecular biology, pathology, and medicine. In particular cases, the present invention concerns antimicrobial compositions and methods related thereto.

### BACKGROUND OF THE INVENTION

The development of antibiotics is considered the most successful story in drug discovery, having saved millions of people's lives since the widespread prescription of penicillin in 1940s. However, according to the World Health Organization (WHO), bacterial infections are still the number one cause of human death, killing ˜6 million people each year worldwide, mostly in developing countries. In addition, drug resistant bacteria have reached epidemic levels during the past few decades. For example, tuberculosis alone causes the deaths of ˜1.6 million people annually, with Mycobacterium tuberculosis, the causative agent, becoming more and more drug resistant: in many countries with a high incidence of tuberculosis (e.g., China), ˜20% cases of newly diagnosed tuberculosis are now resistant to the most widely used drug isoniazid, while the number increases to >45% among previously treated patients.

Even in the United States, bacterial infections have also become a serious threat and burden to public health mainly because of the rising drug resistance. For instance, P. aeruginosa infections account for ˜10 percent of hospital-acquired infections. This Gram-negative bacterium is notorious for its inherited resistance to antibiotics and is therefore a particularly dangerous and dreaded pathogen (Driscoll et al., 2007; Paterson, 2006). Only a few antibiotics are effective, including gentamicin, imipenem, and fluoroquinolones, and even these antibiotics are not effective against all strains. New strains resistant to these antibiotics have continued to emerge. For example, many strains of P. aeruginosa have now acquired metallo-β-lactamase genes and therefore become highly resistant to imipenem (Walsh, 2005; Walsh et al., 2005). Few options are available to treat infections caused by this multiple drug resistant bacterium. Pseudomonas infections are thus a life-threatening disease for patients with cystic fibrosis and severe burns, as well as cancer and AIDS patients who are immuno-compromised.

On the other hand, production of new antibacterial drugs by the pharmaceutical industry has decreased significantly since 1980 (Nathan, 2004). The reasons are complex but may be due mainly to a poor investment yield on anti-infective drugs (Christoffersen, 2006). There is, therefore, an urgent need to find new drugs to combat bacterial infections that are resistant to the current therapies (Nat. Rev. Drug Discov., 2007). In addition, one important strategy to overcome the rising drug resistance is to use combination therapy to treat bacterial infections (Walsh, 2003). The combination of two or more drugs without cross-resistance, which act on different targets, will significantly reduce the likelihood of resistance. However, unfortunately, the common antibiotics such as methicillin and vancomycin have not been used in combination therapy.

Another serious infectious disease is malaria, the so-called “most neglected disease”. Around 2.5 billion people or 40% of the world's population live at risk of malaria, which afflicts about 300-500 million people and kills ˜1.5 million per year. These dreadful numbers will be likely rising mainly because of the increased drug resistance of malaria parasites against commonly used, cheap drugs like chloroquine. In addition, because of the extreme poverty in affected areas, pharmaceutical industry has had little involvement in antimalarial drug discovery/development (Pecoul et al., 1999; Trouiller et al., 2002). For example, during the period 1975-1997 there were 1223 new chemical entities (NCEs) commercialized, of which only 4 (0.3%) are specifically for the treatment of malaria.

DXR is the 2nd enzyme in the non-mevalonate isoprene biosynthesis pathway, as shown in FIG. 1A (Hunter, 2007). This is used by most pathogenic bacteria (except Gram-positive cocci), such as M. tuberculosis, as well as malaria parasites, to make essential isopentenyl diphosphate (IPP) and dimethylallyl diphosphate (DMAPP), which are two common precursors for biosynthesis of all isoprenoids/terpenoids. DXR is essential for the growth of these species. On the other hand, humans and animals use the mevalonate pathway (FIG. 1A) to make IPP and DMAPP, making DXR an attractive drug target for novel anti-infectives. Although considerable progress has been achieved in understanding its biochemical and structural properties during the past decade, fosmidomycin, a naturally occurring antibiotic found in 1980 (Mine et al., 1980; Neu and Kamimura, 1981), together with its close analogs such as FR900098 (FIG. 1B), have been the only potent inhibitors of DXR (Hunter, 2007; Kuzuyama et al., 1998). Indeed, as a potent inhibitor (IC₅₀: 28 nM) of DXR from P. falciparum (Jomaa et al., 1999), fosmidomycin has potent anti-malarial activity, rapidly clearing the parasites from patients' blood (Missinou et al., 2002; Borrmann et al., 2004; Borrmann et al., 2006; Borrmann et al., 2005; Oyakhirome et al., 2007). However, it has a relatively poor pharmacokinetic profile, quickly eliminated from patients' body with a half live in plasma ranging from 0.5-1.5 h. High doses, i.e., 3.6 g/day for two weeks, are therefore required to achieve the desired pharmacological effects.

In addition, fosmidomycin is also a potent inhibitor of DXRs from bacterial species (Kuzuyama et al., 1998; Altincicek et al., 2000; dhiman et al., 2005) and has activity against most gram-negative bacteria such as P. aeruginosa (Mine et al., 1980; Neu and Kamimura, 1981). For example, it inhibits 50% of P. aeruginosa isolates at the minimal inhibition concentration (MIC₅₀) of 6.25 μg/mL, more active than clinically used gentamicin (MIC50: 12.5 μg/mL) (Neu and Kamimura, 1981). It has also excellent activity against many gram-negative bacteria such as E. coli, H. influenzae and Enterobacter sp. However, many strains of these bacteria are now resistant to fosmidomycin. Furthermore, Gram-positive bacteria, such as M. tuberculosis and B. cereus, are generally not sensitive to fosmidomycin. This is attributed to that fosmidomycin, a highly polar and non-lipophilic molecule, is excluded from resistant bacterial cells. Fosmidomycin is transported into the sensitive bacteria and parasites via a glycerol 3-phosphate transporter GlpT. Bacteria lacking GlpT or with a mutant/unfunctional glpT protein are therefore resistant to fosmidomycin by either limited uptake or effective efflux (Dhiman et al., 2005; Brown and Parish, 2008; sakamoto et al,. 2003). Nonetheless, fosmidomycin remains a strong inhibitor of M. tuberculosis DXR with IC₅₀ of 310 nM (Dhiman et al., 2005). Thus, DXR is still a valid target for anti-bacterial drug discovery, so there is a need to find potent inhibitors of bacterial DXRs with good permeability into bacterial cells. These compounds would be a useful, novel class of antibiotics without cross drug resistance.

Another piece of evidence that indicates the need of a new type of DXR inhibitors comes from recent work with T. gondii, which is the causative agent of toxoplasmosis. This protozoan parasite infects most warm-blooded animals, including cats (the primary host) as well as humans. The infection is generally mild for healthy people but can have serious or even fatal effects on a fetus whose mother carries the parasite during pregnancy or on an immuno-compromised person (e.g., HIV, cancer and organ transplant patients). T. gondii is estimated to infect up to ⅓ of world population (Montoya and Liesenfeld, 2004) and the CDC reported that the prevalence of this disease in the US is 11%, including women of childbearing age who are particularly at risk (Jones et al., 2007). Recent study from the Moreno group showed that DXR is essential for the growth of T. gondii, but fosmidomycin has no activity on the parasite. This shows that either it cannot enter into the parasite cells or it is a poor inhibitor of T. gondii DXR. However, in any case, new DXR inhibitors are needed.

DXR catalyzes the isomerization and reduction of 1-deoxy-D-xylulose-5-phosphate (DXP) to 2-C-methyl-D-erythritol 4-phosphate (MEP) in the presence of Mg²⁺ and NADPH, which is a hydride donor (Takahashi et al., 1998), as shown in Scheme 1.

The structure and function of DXR have been actively studied during the past decade and about a dozen of x-ray structures of DXRs from several species (e.g., E. coli and M. tuberculosis), complexed with various combinations of the substrate, inhibitors and cofactors, have been published (Henriksson et al., 2007; Mac Sweeney et al., 2005; Ricagno et al., 2004; Yajima et al., 2007; Yajima et al., 2002; Yajima et al., 2002). The representative quaternary DXR crystal structure (Yajima et al,. 2007) in complex with fosmidomycin, Mg²⁺ and NADPH, is shown in FIG. 2. The Mg²⁺ is coordinated in a distorted octahedral configuration with the two oxygen atoms of hydroxamate, Glu 152 and 231, Asp 150, and a water molecule. The substrate DXP binds to the enzyme at the same site as fosmidomycin, shown superimposed in FIG. 2. The phosph(on)ate group has H-bond and electrostatic interactions with the Lys228 and Ser185 residues. The nicotinamide ring of NADPH is located in a mainly hydrophobic pocket with an orientation that would allow the transfer of a C4 hydride to the substrate.

As a promising anti-infective drug target, much interest has been attracted to develop DXR inhibitors during the past decade (Shtannikov et al., 2007; Yajima et al., 2004; Gottlin et al., 2003; Kuntz et al., 2005; Merckle et al., 2005; Munos et al,. 2008; Ortmann et al., 2007; Silber et al., 2005; Woo et al., 2006). Despite these efforts using either high-throughput screening or medicinal chemistry based on the structures of fosmidomycin/DXP, no other potent DXR inhibitors (IC₅₀s<1 μM) have been identified. This reflects the challenge in discovering potent DXR inhibitors. For example, a high-throughput screening of 32,000 compounds only yielded 30 hits with IC₅₀s of <20 μM (Gottlin et al., 2003). However, the structures of these hits were not disclosed and these compounds therefore cannot be confirmed and further developed.

There is a need in the art to provide additional anti-pathogenic compounds for the treatment of infections, including DXR inhibitors that are useful for antimicrobial therapy.

### BRIEF SUMMARY OF THE INVENTION

The present invention generally concerns methods and compositions for antimicrobial therapy. The antimicrobial therapy may be effective against any kind of microbe, but in specific embodiments the microbe is a bacterium, fungus, protozoan or virus, for example. In specific embodiments, the microbe is a bacterium. In particular cases, the microbe has the enzyme 1-Deoxy-D-xylulose-5-phosphate reductoisomerase (DXR) and the antimicrobial composition targets DXR, although in alternative embodiments the microbe lacks DXR but the composition is still effective against the microbe. In some cases, the antimicrobial composition is effective against one or more microbes that are resistant to one or more other antimicrobial therapies. In some cases, the antimicrobial agent comprises an electron-deficient hydrophobic group that has interacts with Trp211 of DXR. In specific embodiments, the compound contains electron-deficient heterocyclic rings that specifically interact with the electron-rich indole ring of Trp211, for example. In certain aspects, the compositions comprise a phosphate group, a pyridine group, and a hydroxymate group.

Exemplary microbes that have DXR include but are not limited to Mycobacterium tuberculosis, Helicobacter pylori, Listeria monozytogenes, Escherichia coli, Pseudomonas aeruginosa, Haemophilus influenzae, Bacillus cereus, Toxoplasma gondii, and Bacillus subtilis.

In specific embodiments, the antimicrobial agents are effective against one or more bacteria selected from the group consisting of the following phyla: 1) Aquificae; 2) Xenobacteria; 3) Fibrobacter; 4) Bacteroids; 5) Firmicutes; 6) Planctomycetes; 7) Chrysogenetic; 8) Cyanobacteria; 9) Thermomicrobia; 10) Chlorobia; 11) Proteobacteria; 12) Spirochaetes; 13) Flavobacteria; 14) Fusobacteria; and 15) Verrucomicrobia. In specific cases, the disinfectants of the present invention are useful against Gram negative cocci; Gram positive bacilli; Gram negative bacilli, Spirochaetes, Rickettsia, and Mycoplasma.

In certain cases, the antimicrobial agents are useful against Corynebacterium, Listeria, Bacillus, Clostridium, Neisseria, Enterobacteria, E. coli, Salmonella, Shigella, Campylobacter, Chlamydia, Borrelia, Francisella, Leptospira, Treponema, Proteus, Yersinia pestis, Vibrio, Helicobacter, Haemophila, Bordetella, Brucella, and Bacteriodes. In particular cases, the disinfectants are useful against Listeria monocytogenes, Clostridium botulinum, Legionella pneumophila, E. coli, Salmonella enterica, Neisseria meningitides, Yersinia pestis, Mycobacterium tuberculosis, Vibrio cholera, Group A hemolytic streptococei, Diplococcus pneumonia, Moraxella catarrhalis, Neisseria gonorrhoeae, C. jeikeium, Mycobacterium avium complex, M. kansasii, M. leprae, M. tuberculosis, Nocardia sp, Acinetobacter calcoaceticus, Flavobacterium meningosepticum, Pseudomonas aeruginosa, P. alcaligenes, other Pseudomonas sp, Stenotrophomonas maltophilia, Brucella, Bordetella, Francisella, Legionella spp, Leptospira sp, Bacteroides fragilis, other Bacteroides sp, Fusobacterium sp, Prevotella sp, Veillonella sp, Peptococcus niger, Peptostreptococcus sp, Actinomyces, Bifidobacterium, Eubacterium, and Propionibacterium spp, Clostridium botulinum, C. perfringens, C. tetani, other Clostridium sp, Staphylococcus aureus (coagulase-positive), S. epidermidis (coagulase-negative), other coagulase-negative staphylococci, Enterococcus faecalis, E. faecium, Streptococcus agalactiae (group B streptococcus), S. bovis, S. pneumoniae, S. pyogenes (group A streptococcus), viridans group streptococci (S. mutans, S. mitis, S. salivarius, S. sanguis), S. anginosus group (S. anginosus, S. milleri, S. constellatus), Gemella morbillorum. Bacillus anthracis, Erysipelothrix rhusiopathiae, Gardnerella vaginalis (gram-variable), Enterobacteriaceae (Citrobacter sp, Enterobacter aerogenes, Escherichia coli, Klebsiella sp, Morganella morganii, Proteus sp, Providencia rettgeri, Salmonella typhi, other Salmonella sp, Serratia marcescens, Shigella sp, Yersinia enterocolitica, Y. pestis), Aeromonas hydrophila, Chromobacterium violaceum, Pasturella multocida, Plesiomonas shigelloides, Actinobacillus actinomycetemcomitans, Bartonella bacilliformis, B. henselae, B. quintana, Eikenella corrodens, Haemophilus influenzae, other Haemophilus sp, Mycoplasma pneumonia, Borrelia burgdorferi, Treponema pallidum Campylobacter jejuni, Helicobacter pylori, Vibrio cholerae, V. vulnificus, Chlamydia trachomatis, Chlamydophila pneumoniae, C. psittaci, Coxiella burnetii, Rickettsia prowazekii, R. rickettsii, R. typhi, R. tsutsugamushi, R. africae, R. akari, Ehrlichia canis, Ehrlichia chaffeensis, and Anaplasma phagocytophilum.

In particular embodiments of the present invention, the antimicrobial agent is effective against one or more Apicomplexa protozoa (including Aconoidasida and Conoidasida), including one or more pathogenic parasites. Exemplary Acicomplexa genera include Aggregata, Atoxoplasma, Cystoisospora, Schellackia, Toxoplasma, Akiba, Babesiosoma, Babesia, Haemogregarina, Haemoproteus, Hepatozoon, Karyolysus, Leucocytozoon, Plasmodium, Sarcocystis and Theileria.

In specific embodiments, the antimicrobial agent is effective against one or more parasites selected from the group consisting of Plasmodium, Babesium, coccidium, Cryptosporidium, Toxoplasma, Cyclospora and Isospora.

In certain embodiments of the invention, the antimicrobial therapy comprises one or more compositions encompassed by the invention. The antimicrobial composition may be formulated in a pharmaceutical composition. In specific embodiments, the composition is administered to an individual that has an infection of the microbe, has been exposed to the microbe, or that may be exposed to the microbe. In certain embodiments, the antimicrobial therapy of the invention is given to an individual that will receive, is receiving, or has received another therapy for the microbe. In specific cases, the effective composition is preventative of infection of the microbe.

In particular cases, the antimicrobial composition is delivered to a mammal, including a human, dog, cat, horse, goat, sheep, cow, or pig. In specific embodiments, the antimicrobial composition is delivered systemically or non-systemically. The composition may be delivered by injection, topically, or orally, for example. The composition may be delivered to the individual in a single dose or in multiple doses. Multiples doses may be delivered over the course of a single day, over the course of two or more days, one week, two weeks, or more.

Embodiments of the present invention include methods of producing the compositions of the invention and methods of treating and/or preventing infection in an individual.

Other and further objects, features, and advantages would be apparent and eventually more readily understood by reading the following specification and be reference to the accompanying drawings forming a part thereof, or any examples of the presently preferred embodiments of the invention given for the purpose of the disclosure.

### BRIEF DESCRIPTION OF THE DRAWINGS

For a more complete understanding of the present invention, reference is now made to the following descriptions taken in conjunction with the accompanying drawing, in which:

FIG. 1 shows two distinct isoprene biosynthesis pathways.

FIG. 2 illustrates exemplary highly potent PfDXR inhibitors and a known structure.

FIG. 3 also demonstrates structures of exemplary DXR inhibitors.

FIG. 4 (A) QSAR electron-withdrawing fields superimposed with the aligned structures of 1 and 3, with the red boxes being favorable; (B) Active site of the superimposed crystal structures of EcDXR:1 and EcDXR:3 complexes. Mg²⁺ is shown as a pink sphere.

FIG. 5 provides a general synthesis for exemplary compounds.

FIG. 6 shows a gel image of FPLC (Superdex 75) fractions containing purified PfDXR. The central three fractions (labeled) were used for enzyme activity/inhibition assay and crystallization.

FIG. 7 demonstrates exemplary DXR inhibitors of the invention.

FIG. 8 illustrates exemplary procedures to synthesize the inhibitors of the invention.

FIG. 9 provides illustrations of exemplary compounds of the invention.

FIG. 10 provides a clustal X alignment of E. coli (Ec), T. gondii (Tg) and P. falciparum (Pf) DXR.

FIG. 11 shows (A) effects of [Mg²⁺] on TgDXR catalyzed reaction. See Example 6 for a general assay condition; (B) effects of divalent metal ions on TgDXR catalyzed reaction; (C) Effects of pH on TgDXR catalyzed reaction ([Mg²⁺]=2 mM); (D) Effects of [DXP] on TgDXR catalyzed reaction ([Mg²⁺]=4 mM).

FIG. 12 shows correlations between TgDXR inhibition and that of (A) EcDXR and (B) PfDXR.

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The present invention has utilized a combination of traditional medicinal chemistry and computational, structure based drug design to develop novel small molecule inhibitors of 1-deoxy-D-xylulose-5-phosphate reductoisomerase (DXR) whose activity may be tested in vitro on pathogenic bacteria and parasites. DXR is a validated target for anti-infective drug discovery. The present invention provides novel inhibitors that are clinically useful anti-infective drugs to treat bacterial infections, malaria and other parasitic diseases, caused by, e.g., Pseudomonas aeruginosa, Mycobacterium tuberculosis, Plasmodium falciparum and Toxoplasma gondii.

Isoprene biosynthesis is essential to all organisms. Humans and animals use the mevalonate pathway to produce isopentenyl diphosphate (IPP) and dimethylallyl diphosphate (DMAPP), two common precursors for all isoprenoid biosynthesis; however, in most pathogenic bacteria, such as P. aeruginosa and M. tuberculosis, as well as apicomplexan parasites, such as P. falciparum and T. gondii, the non-mevalonate pathway, or 2C-methyl-D-erythritol-4-phosphate (MEP) pathway, is used to make IPP and DMAPP (Hunter, 2007). Since humans lack all the 7 enzymes in the non-mevalonate pathway, it has become an attractive target for anti-infective drug discovery (Rodriguez-Concepcion, 2004; Singh et al., 2007; Testa and Brown, 2003). Fosmidomycin has been found to be the only potent inhibitor of this pathway, blocking DXR, the 2nd enzyme, and has antibacterial activity against many Gram-negative bacteria (Mine et al., 1980; Neu and Kamimura, 1981) and antimalarial activity in recent clinical trials (Missinou et al., 2002; Borrmann et al., 2004; Borrmann et al., 2006; Borrmann et al., 2005; Oyakhirome et al., 2007). However, Gram-positive bacteria (e.g., M. tuberculosis) and some Gram-negative bacteria (Shtannikov et al., 2007) as well as certain pathogenic parasites (e.g., T. gondii) are resistant to fosmidomycin. In addition, it has a poor pharmacokinetic profile with a half-life in plasma of 0.5-1.5 h. Given the current devastating situation facing quickly rising drug resistance as well as shortage of new anti-infective drugs, there is a pressing need to find new weaponry for infectious diseases. Based on rational, structure based design, the present invention provides a submicromolar inhibitor of DXR with a distinct structure from that of fosmidomycin.

In specific aspects, a combination of traditional medicinal chemistry and computational, structure based drug design is used to develop novel inhibitors of 1-deoxy-D-xylulose-5-phosphate reductoisomerase (DXR). Since fosmidomycin is the only potent DXR inhibitor but, due to its very polar structure and poor pharmacokinetic properties, it has no activity against many bacteria and pathogenic parasites, novel, more lipophilic DXR inhibitors are now needed. Based on rational, structure based design, the inventors have found novel, drug-like lead inhibitors with Kᵢs as low as 310 nM against a recombinant E. coli DXR enzyme. The docking studies showed that they could bind to DXR in a different mode from that of fosmidomycin. In development of the present invention, there is 1) use of medicinal chemistry to make several series of compound libraries based on the scaffold of the lead inhibitor, to find compounds with improved activity; 2) carrying out of quantitative structure activity relationship (QSAR) studies of these compounds; 3) obtaining of x-ray crystal structures of DXR in complex with novel inhibitors; and 4) use of the results from the computational and crystallographic studies to characterize further drug design and synthesis.

One can test in vitro biological activities of lead inhibitors as well as potent inhibitors. In some cases, a recombinant E. coli DXR is used as a primary screen. Good inhibitors against the E. coli enzyme are further tested against DXRs from M. tuberculosis, P. falciparum and T. gondii, for example, in order to obtain an inhibition/selectivity profile of novel DXR inhibitors. Next, one can test the activities of these DXR inhibitors on a broad range of bacteria as well as apicomplexan parasites, including E. coli, P. aeruginosa, Haemophilus influenzae, Bacillus subtilis, Bacillus cereus, M. tuberculosis, P. falciparum and T. gondii, for example These species include 3 Gram-negative, 3 Gram-positive bacteria and 2 eukaryotic parasites, with several being notorious pathogens that are responsible for deaths of millions of people each year. Finally, one can also test the cytotoxicity of potent DXR inhibitors on mammalian cell lines (e.g., 3T3) to evaluate their potential toxicity.

### I. Exemplary Chemical Compositions

In particular embodiments of the invention, the DXR inhibitor comprises the following general structure:

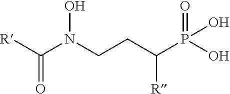

wherein

R′ is Methyl (Me) or Hydrogen (H). In some embodiments, R″ comprises a nitrogen containing functional group. In certain embodiments, R″ comprises an amino, substituted amino, imino, heterocyclyl, and/or substituted heterocyclyl functional group. In some embodiments, R″ comprises a fuctional groups selected from the group consisting of azetidinyl, tetrazoyl, piperidinyl, piperazinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, indanyl, isoxazolyl, isoxazqlidinyl, morpholinyl, thiazolyl, thiazolinyl, thiazolidinyl, isothiazolyl, and isothiazolidinyl.

In particular embodiments of the invention, R″ is selected from the group consisting of

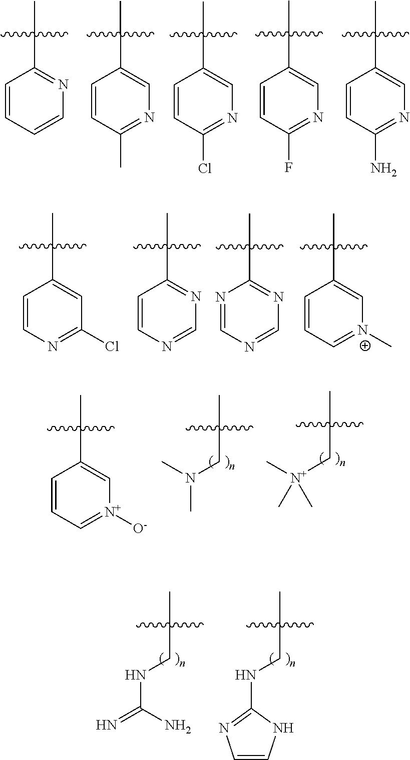

wherein n=0-2.

II. Exemplary Chemical Group Definitions

In embodiments of the invention, one can make modifications to the compounds described herein. The following addresses exemplary modifications.

When used in the context of a chemical group, “hydrogen” means —H; “hydroxy” means —OH; “oxo” means ═O; “halo” means independently —F, —Cl, —Br or —I; “amino” means —NH₂ (see below for definitions of groups containing the term amino, e.g., alkylamino); “substituted amino,” refers to the group —NHR, —NRR, —N⁺RRR where each R is independently selected from the group: optionally substituted alkyl, optionally substituted alkoxy, optionally substituted aryl, optionally substituted heterocyclyl, acyl, carboxy, alkoxycarbonyl, sulfanyl, sulfinyl and sulfonyl, e.g., diethylamino, methylsulfonylamino, furanyl-oxy-sulfonamino; “hydroxyamino” means —NHOH; “nitro” means —NO₂; imino means ═NH (see below for definitions of groups containing the term imino, e.g., alkylimino); “cyano” means —CN; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; “thio” means ═S; “thioether” means —S—; “sulfonamido” means —NHS(O)₂— (see below for definitions of groups containing the term sulfonamido, e.g., alkylsulfonamido); “sulfonyl” means —S(O)₂— (see below for definitions of groups containing the term sulfonyl, e.g., alkylsulfonyl); “sulfinyl” means —S(O)— (see below for definitions of groups containing the term sulfinyl, e.g., alkylsulfinyl); and “silyl” means —SiH₃ (see below for definitions of group(s) containing the term silyl, e.g., alkylsilyl).

The symbol “—” means a single bond, “═” means a double bond, and “≡” means triple bond. The symbol “ [symbol] ” represents a single bond or a double bond. The symbol “ [symbol] ”, when drawn perpendicularly across a bond indicates a point of attachment of the group. It is noted that the point of attachment is typically only identified in this manner for larger groups in order to assist the reader in rapidly and unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the conformation is unknown (e.g., either R or S), the geometry is unknown (e.g., either E or Z) or the compound is present as mixture of conformation or geometries (e.g., a 50%/50% mixture).

When a group “R” is depicted as a “floating group” on a ring system, for example, in the formula:

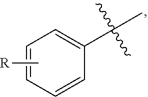

then R may replace any hydrogen atom attached to any of the ring atoms, including a depicted, implied, or expressly defined hydrogen, so long as a stable structure is formed.

When a group “R” is depicted as a “floating group” on a fused ring system, as for example in the formula:

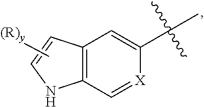

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

When y is 2 and “(R)_{y}” is depicted as a floating group on a ring system having one or more ring atoms having two replaceable hydrogens, e.g., a saturated ring carbon, as for example in the formula:

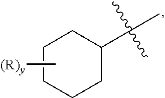

then each of the two R groups can reside on the same or a different ring atom. For example, when R is methyl and both R groups are attached to the same ring atom, a geminal dimethyl group results. Where specifically provided for, two R groups may be taken together to form a divalent group, such as one of the divalent groups further defined below. When such a divalent group is attached to the same ring atom, a spirocyclic ring structure will result.

When the point of attachment is depicted as “floating”, for example, in the formula:

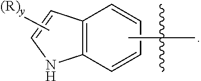

then the point of attachment may replace any replaceable hydrogen atom on any of the ring atoms of either of the fused rings unless specified otherwise.

In the case of a double-bonded R group (e.g., oxo, imino, thio, alkylidene, etc.), any pair of implicit or explicit hydrogen atoms attached to one ring atom can be replaced by the R group. This concept is exemplified below:

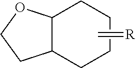

represents

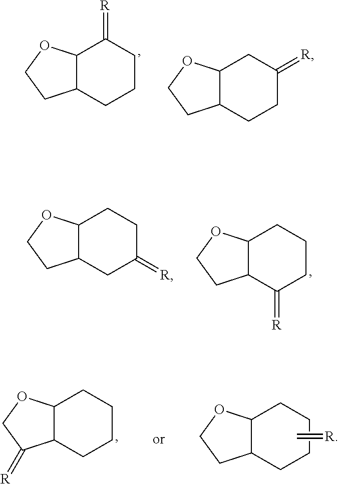

For the groups and classes below, the following parenthetical subscripts further define the group/class as follows: “(Cn)” defines the exact number (n) of carbon atoms in the group/class. “(C≦n)” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. For example, “alkoxy_{(C≦10)}” designates those alkoxy groups having from 1 to 10 carbon atoms (e.g., 1, 2, 3, 4, 5, 6, 7, 8, 9, or 10, or any range derivable therein (e.g., 3 to 10 carbon atoms). (Cn-n′) defines both the minimum (n) and maximum number (n′) of carbon atoms in the group. Similarly, “alkyl_{(C2-10)}” designates those alkyl groups having from 2 to 10 carbon atoms (e.g., 2, 3, 4, 5, 6, 7, 8, 9, or 10, or any range derivable therein (e.g., 3 to 10 carbon atoms)).

The term “alkyl” when used without the “substituted” modifier refers to a non-aromatic monovalent group with a saturated carbon atom as the point of attachment, a linear or branched, cyclo, cyclic or acyclic structure, no carbon-carbon double or triple bonds, and no atoms other than carbon and hydrogen. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, cyclohexyl, and cyclohexylmethyl are non-limiting examples of alkyl groups. The term “substituted alkyl” refers to a non-aromatic monovalent group with a saturated carbon atom as the point of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkyl groups: —CH₂OH, —CH₂Cl, —CH₂Br, —CH₂SH, —CF₃, —CH₂CN, —CH₂C(O)H, —CH₂C(O)OH, —CH₂C(O)OCH₃, —CH₂C(O)NH₂, —CH₂C(O)NHCH₃, —CH₂C(O)CH₃, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, —CH₂CF₃, —CH₂CH₂OC(O)CH₃, —CH₂CH₂NHCO₂C(CH₃)₃, and —CH₂Si(CH₃)₃.

The term “alkanediyl” when used without the “substituted” modifier refers to a non-aromatic divalent group, wherein the alkanediyl group is attached with two a-bonds, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, —CH₂CH₂CH₂—, and

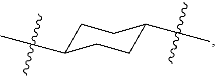 [SMILES: *C1CCC(*)CC1]

are non-limiting examples of alkanediyl groups. The term “substituted alkanediyl” refers to a non-aromatic monovalent group, wherein the alkynediyl group is attached with two σ-bonds, with one or two saturated carbon atom(s) as the point(s) of attachment, a linear or branched, cyclo, cyclic or acyclic structure, no carbon-carbon double or triple bonds, and at least one atom independently selected from the group consisting of N, O, F, Cl, Br, I, Si, P, and S. The following groups are non-limiting examples of substituted alkanediyl groups: —CH(F)—, —CF₂—, —CH(Cl)—, —CH(OH)—, —CH(OCH₃)—, and —CH₂CH(Cl)—.

The term “alkane” when used without the “substituted” modifier refers to a non-aromatic hydrocarbon consisting only of saturated carbon atoms and hydrogen and having a linear or branched, cyclo, cyclic or acyclic structure. Thus, as used herein cycloalkane is a subset of alkane. The compounds CH₄ (methane), CH₃CH₃ (ethane), CH₃CH₂CH₃ (propane), (CH₂)₃ (cyclopropane), CH₃CH₂CH₂CH₃ (n-butane), and CH₃CH(CH₃)CH₃ (isobutane), are non-limiting examples of alkanes. A “substituted alkane” differs from an alkane in that it also comprises at least one atom independently selected from the group consisting of N, O, F, Cl, Br, I, Si, P, and S. The following compounds are non-limiting examples of substituted alkanes: CH₃OH, CH₃Cl, nitromethane, CF₄, CH₃OCH₃ and CH₃CH₂NH₂.

The term “alkenyl” when used without the “substituted” modifier refers to a monovalent group with a nonaromatic carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “substituted alkenyl” refers to a monovalent group with a nonaromatic carbon atom as the point of attachment, at least one nonaromatic carbon-carbon double bond, no carbon-carbon triple bonds, a linear or branched, cyclo, cyclic or acyclic structure, and at least one atom independently selected from the group consisting of N, O, F, Cl, Br, I, Si, P, and S. The groups, —CH═CHF, —CH═CHCl and —CH═CHBr, are non-limiting examples of substituted alkenyl groups.

The term “alkenediyl” when used without the “substituted” modifier refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, —CH═CH—, —CH═C(CH₃)CH₂—, —CH═CHCH₂—, and

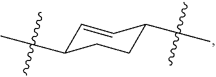 [SMILES: *C1C=CC(*)CC1]

are non-limiting examples of alkenediyl groups. The term “substituted alkenediyl” refers to a non-aromatic divalent group, wherein the alkenediyl group is attached with two σ-bonds, with two carbon atoms as points of attachment, a linear or branched, cyclo, cyclic or acyclic structure, at least one nonaromatic carbon-carbon double bond, no carbon-carbon triple bonds, and at least one atom independently selected from the group consisting of N, O, F, Cl, Br, I, Si, P, and S. The following groups are non-limiting examples of substituted alkenediyl groups: —CF═CH—, —C(OH)═CH—, and —CH₂CH═C(Cl)—.

The term “alkene” when used without the “substituted” modifier refers to a non-aromatic hydrocarbon having at least one carbon-carbon double bond and a linear or branched, cyclo, cyclic or acyclic structure. Thus, as used herein, cycloalkene is a subset of alkene. The compounds C₂H₄ (ethylene), CH₃CH═CH₂ (propene) and cylcohexene are non-limiting examples of alkenes. A “substituted alkene” differs from an alkene in that it also comprises at least one atom independently selected from the group consisting of N, O, F, Cl, Br, I, Si, P, and S.

The term “alkynyl” when used without the “substituted” modifier refers to a monovalent group with a nonaromatic carbon atom as the point of attachment, a linear or branched, cyclo, cyclic or acyclic structure, at least one carbon-carbon triple bond, and no atoms other than carbon and hydrogen. The groups, —C≡CH, —C≡CCH₃, —C≡CC₆H₅ and —CH₂C≡CCH₃, are non-limiting examples of alkynyl groups. The term “substituted alkynyl” refers to a monovalent group with a nonaromatic carbon atom as the point of attachment and at least one carbon-carbon triple bond, a linear or branched, cyclo, cyclic or acyclic structure, and at least one atom independently selected from the group consisting of N, O, F, Cl, Br, I, Si, P, and S. The group, —C≡CSi(CH₃)₃, is a non-limiting example of a substituted alkynyl group.

The term “alkynediyl” when used without the “substituted” modifier refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and no atoms other than carbon and hydrogen. The groups, —C≡C—, —C≡CCH₂—, and —C≡CCH(CH₃)— are non-limiting examples of alkynediyl groups. The term “substituted alkynediyl” refers to a non-aromatic divalent group, wherein the alkynediyl group is attached with two σ-bonds, with two carbon atoms as points of attachment, a linear or branched, cyclo, cyclic or acyclic structure, at least one carbon-carbon triple bond, and at least one atom independently selected from the group consisting of N, O, F, Cl, Br, I, Si, P, and S. The groups —C≡CCFH— and —C≡CHCH(Cl)— are non-limiting examples of substituted alkynediyl groups.

The term “alkyne” when used without the “substituted” modifier refers to a non-aromatic hydrocarbon having at least one carbon-carbon triple bond and a linear or branched, cyclo, cyclic or acyclic structure. Thus, as used herein, cycloalkene is a subset of alkene. The compounds C₂H₂ (acetylene), CH₃C≡CH (propene) and cylcooctyne are non-limiting examples of alkenes. A “substituted alkene” differs from an alkene in that it also comprises at least one atom independently selected from the group consisting of N, O, F, Cl, Br, I, Si, P, and S.

The term “aryl” when used without the “substituted” modifier refers to a monovalent group with an aromatic carbon atom as the point of attachment, said carbon atom forming part of one or more six-membered aromatic ring structure(s) wherein the ring atoms are all carbon, and wherein the monovalent group consists of no atoms other than carbon and hydrogen. Non-limiting examples of aryl groups include phenyl (Ph), methylphenyl, (dimethyl)phenyl, —C₆H₄CH₂CH₃ (ethylphenyl), —C₆H₄CH₂CH₂CH₃ (propylphenyl), —C₆H₄CH(CH₃)₂, —C₆H₄CH(CH₂)₂, —C₆H₃(CH₃)CH₂CH₃ (methylethylphenyl), —C₆H₄CH═CH₂ (vinylphenyl), —C₆H₄CH═CHCH₃, —C₆H₄C≡CH, —C₆H₄C≡CCH₃, naphthyl, and the monovalent group derived from biphenyl. The term “substituted aryl” refers to a monovalent group with an aromatic carbon atom as the point of attachment, said carbon atom forming part of one or more six-membered aromatic ring structure(s) wherein the ring atoms are all carbon, and wherein the monovalent group further has at least one atom independently selected from the group consisting of N, O, F, Cl, Br, I, Si, P, and S. Non-limiting examples of substituted aryl groups include the groups: —C₆H₄F, —C₆H₄Cl, —C₆H₄Br, —C₆H₄I, —C₆H₄OH, —C₆H₄OCH₃, —C₆H₄OCH₂CH₃, —C₆H₄OC(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, and —C₆H₄CON(CH₃)₂.

“Arylalkyl” refers to a residue in which an aryl moiety is attached to a parent structure via one of an alkylene, alkylidene, or alkylidyne. Examples include benzyl, phenethyl, phenylvinyl, phenylallyl and the like. The aryl, alkylene, alkylidene, or alkylidyne portion of an arylalkyl group may be optionally substituted. “Lower arylalkyl” refers to an arylalkyl where the “alkyl” portion of the group has one to eight carbons.

The term “arenediyl” when used without the “substituted” modifier refers to a divalent group, wherein the arenediyl group is attached with two σ-bonds, with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. Non-limiting examples of arenediyl groups include:

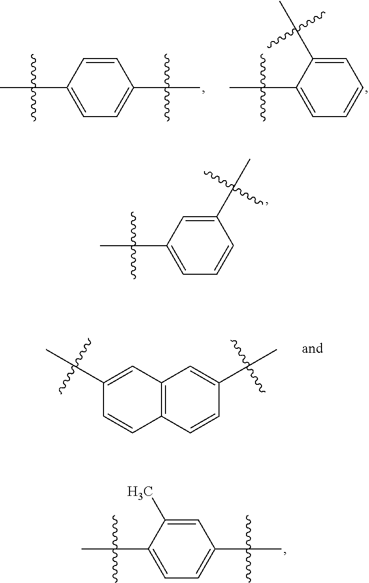

The term “substituted arenediyl” refers to a divalent group, wherein the arenediyl group is attached with two σ-bonds, with two aromatic carbon atoms as points of attachment, said carbon atoms forming part of one or more six-membered aromatic rings structure(s), wherein the ring atoms are carbon, and wherein the divalent group further has at least one atom independently selected from the group consisting of N, O, F, Cl, Br, I, Si, P, and S.

The term “arene” when used without the “substituted” modifier refers to an hydrocarbon having at least one six-membered aromatic ring. One or more alkyl, alkenyl or alkynyl groups may be optionally attached to this ring. Also this ring may optionally be fused with other rings, including non-aromatic rings. Benzene, toluene, naphthalene, and biphenyl are non-limiting examples of arenes. A “substituted arene” differs from an arene in that it also comprises at least one atom independently selected from the group consisting of N, O, F, Cl, Br, I, Si, P, and S. Phenol and nitrobenzene are non-limiting examples of substituted arenes.

The term “aralkyl” when used without the “substituted” modifier refers to the monovalent group -alkanediyl-aryl, in which the terms alkanediyl and aryl are each used in a manner consistent with the definitions provided above. Non-limiting examples of aralkyls are: phenylmethyl (benzyl, Bn), 1-phenyl-ethyl, 2-phenyl-ethyl, indenyl and 2,3-dihydro-indenyl, provided that indenyl and 2,3-dihydro-indenyl are only examples of aralkyl in so far as the point of attachment in each case is one of the saturated carbon atoms. When the term “aralkyl” is used with the “substituted” modifier, either one or both the alkanediyl and the aryl is substituted. Non-limiting examples of substituted aralkyls are: (3-chlorophenyl)-methyl, 2-oxo-2-phenyl-ethyl (phenylcarbonylmethyl), 2-chloro-2-phenyl-ethyl, chromanyl where the point of attachment is one of the saturated carbon atoms, and tetrahydroquinolinyl where the point of attachment is one of the saturated atoms.

The term “heteroaryl” when used without the “substituted” modifier refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group consists of no atoms other than carbon, hydrogen, aromatic nitrogen, aromatic oxygen and aromatic sulfur. Non-limiting examples of aryl groups include acridinyl, furanyl, imidazoimidazolyl, imidazopyrazolyl, imidazopyridinyl, imidazopyrimidinyl, indolyl, indazolinyl, methylpyridyl, oxazolyl, phenylimidazolyl, pyridyl, pyrrolyl, pyrimidyl, pyrazinyl, quinolyl, quinazolyl, quinoxalinyl, tetrahydroquinolinyl, thienyl, triazinyl, pyrrolopyridinyl, pyrrolopyrimidinyl, pyrrolopyrazinyl, pyrrolotriazinyl, pyrroloimidazolyl, chromenyl (where the point of attachment is one of the aromatic atoms), and chromanyl (where the point of attachment is one of the aromatic atoms). The term “substituted heteroaryl” refers to a monovalent group with an aromatic carbon atom or nitrogen atom as the point of attachment, said carbon atom or nitrogen atom forming part of an aromatic ring structure wherein at least one of the ring atoms is nitrogen, oxygen or sulfur, and wherein the monovalent group further has at least one atom independently selected from the group consisting of non-aromatic nitrogen, non-aromatic oxygen, non aromatic sulfur F, Cl, Br, I, Si, and P.

“Heterocyclyl” refers to a stable 3- to 15-membered ring that consists of carbon atoms and from one to five heteroatoms selected from the group consisting of nitrogen, phosphorus, oxygen and sulfur. For purposes of this invention, the heterocyclyl ring may be a monocyclic, bicyclic or tricyclic ring system, which may include fused or bridged ring systems, either aromatic, saturated, or combinations thereof; and the nitrogen, phosphorus, carbon or sulfur atoms in the hetemyclyl ring may be optionally oxidized to various oxidation states, for example for the purposes of this invention and to negate undo repetition in the description the corresponding N-oxide of pyridine derivatives, and the like, are understood to be included as compounds of the invention. In addition, the nitrogen atom may be optionally quaternized; and the ring may be partially or fully saturated or aromatic. Examples of such heterocyclyl rings include, but are not limited to, azetidinyl, acridinyl, benzodioxolyl, benzodioxanyl, benzofuranyl, carbazoyl, cinnolinyl, dioxoianyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyi, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, indanyl, isoxazolyl, isoxazqlidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, tetrahydmfuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

The term “heteroarenediyl” when used without the “substituted” modifier refers to a divalent group, wherein the heteroarenediyl group is attached with two σ-bonds, with an aromatic carbon atom or nitrogen atom as the point of attachment, said carbon atom or nitrogen atom forming part of one or more aromatic ring structure(s) wherein at least one of the ring atoms is nitrogen, oxygen or sulfur, and wherein the divalent group consists of no atoms other than carbon, hydrogen, aromatic nitrogen, aromatic oxygen and aromatic sulfur. Non-limiting examples of heteroarenediyl groups include:

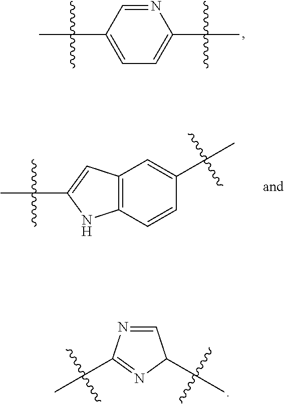 [SMILES: *c1ccc(*)nc1 ; *c1ccc2[nH]c(*)cc2c1 ; *C1=NC(*)C=N1]

Specific examples of heteroarenediyl groups contemplated by the present disclosure include, but are not limited to purine, quinoline, quninolinium, pyridine, pyridinium, pyrimidine, imidazole, pyrazine, triazole, 1,2,3-triazole, 1,2,4-triazone and derivatives thereof.

The term “substituted heteroarenediyl” refers to a divalent group, wherein the heteroarenediyl group is attached with two a-bonds, with an aromatic carbon atom or nitrogen atom as points of attachment, said carbon atom or nitrogen atom forming part of one or more six-membered aromatic ring structure(s), wherein at least one of the ring atoms is nitrogen, oxygen or sulfur, and wherein the divalent group further has at least one atom independently selected from the group consisting of non-aromatic nitrogen, non-aromatic oxygen, non aromatic sulfur F, Cl, Br, I, Si, and P. Specific examples of substituted heteroarenediyl groups contemplated by the present disclosure include, but are not limited to purine, quinoline, quninolinium, pyridine, pyridinium, pyrimidine, imidazole, pyrazine, triazole, 1,2,3-triazole, 1,2,4-triazone and derivatives thereof. In some examples, the substituted heteroarenediyl is functionalized by an electron withdrawing group. Particular examples of electrion withdrawing groups include, but are not limited to —Cl, —F, —Br, —NO₂, —COOR (carboxylate), —COR (acyl), —CN, —SO₂R (sulfone), —SO₂NR₁R₂ (sulfamde), —P(O)(OR)₂ wherein R, R₁, and R₂ are independently selected from alkyl, alkoxy, alkene, etc.

The term “heteroaralkyl” when used without the “substituted” modifier refers to the monovalent group -alkanediyl-heteroaryl, in which the terms alkanediyl and heteroaryl are each used in a manner consistent with the definitions provided above. Non-limiting examples of aralkyls are: pyridylmethyl, and thienylmethyl. When the term “heteroaralkyl” is used with the “substituted” modifier, either one or both the alkanediyl and the heteroaryl is substituted.

The term “acyl” when used without the “substituted” modifier refers to a monovalent group with a carbon atom of a carbonyl group as the point of attachment, further having a linear or branched, cyclo, cyclic or acyclic structure, further having no additional atoms that are not carbon or hydrogen, beyond the oxygen atom of the carbonyl group. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)C₆H₄CH₂CH₃, —COC₆H₃(CH₃)₂, and —C(O)CH₂C₆H₅, are non-limiting examples of acyl groups. The term “acyl” therefore encompasses, but is not limited to groups sometimes referred to as “alkyl carbonyl” and “aryl carbonyl” groups. The term “substituted acyl” refers to a monovalent group with a carbon atom of a carbonyl group as the point of attachment, further having a linear or branched, cyclo, cyclic or acyclic structure, further having at least one atom, in addition to the oxygen of the carbonyl group, independently selected from the group consisting of N, O, F, Cl, Br, I, Si, P, and S. The groups, —C(O)CH₂CF₃, —CO₂H (carboxyl), —CO₂CH₃ (methylcarboxyl), —CO₂CH₂CH₃, —CO₂CH₂CH₂CH₃, —CO₂C₆H₅, —CO₂CH(CH₃)₂, —CO₂CH(CH₂)₂, —C(O)NH₂ (carbamoyl), —C(O)NHCH₃, —C(O)NHCH₂CH₃, —CONHCH(CH₃)₂, —CONHCH(CH₂)₂, —CON(CH₃)₂, —CONHCH₂CF₃, —CO-pyridyl, —CO-imidazoyl, and —C(O)N₃, are non-limiting examples of substituted acyl groups. The term “substituted acyl” encompasses, but is not limited to, “heteroaryl carbonyl” groups.

The term “alkylidene” when used without the “substituted” modifier refers to the divalent group ═CRR′, wherein the alkylidene group is attached with one σ-bond and one π-bond, in which R and R′ are independently hydrogen, alkyl, or R and R′ are taken together to represent alkanediyl. Non-limiting examples of alkylidene groups include: ═CH₂, αCH(CH₂CH₃), and ═C(CH₃)₂. The term “substituted alkylidene” refers to the group ═CRR′, wherein the alkylidene group is attached with one σ-bond and one π-bond, in which R and R′ are independently hydrogen, alkyl, substituted alkyl, or R and R′ are taken together to represent a substituted alkanediyl, provided that either one of R and R′ is a substituted alkyl or R and R′ are taken together to represent a substituted alkanediyl.

The term “alkoxy” when used without the “substituted” modifier refers to the group —OR, in which R is an alkyl, as that term is defined above. Non-limiting examples of alkoxy groups include: —OCH₃, —OCH₂CH₃, —OCH₂CH₂CH₃, —OCH(CH₃)₂, —OCH(CH₂)₂, —O-cyclopentyl, and —O-cyclohexyl. The term “substituted alkoxy” refers to the group —OR, in which R is a substituted alkyl, as that term is defined above. For example, —OCH₂CF₃ is a substituted alkoxy group.

The term “alcohol” when used without the “substituted” modifier corresponds to an alkane, as defined above, wherein at least one of the hydrogen atoms has been replaced with a hydroxy group. Alcohols have a linear or branched, cyclo, cyclic or acyclic structure. The compounds methanol, ethanol and cyclohexanol are non-limiting examples of alcohols. A “substituted alkane” differs from an alcohol in that it also comprises at least one atom independently selected from the group consisting of N, F, Cl, Br, I, Si, P, and S.

Similarly, the terms “alkenyloxy”, “alkynyloxy”, “aryloxy”, “aralkoxy”, “heteroaryloxy”, “heteroaralkoxy” and “acyloxy”, when used without the “substituted” modifier, refers to groups, defined as —OR, in which R is alkenyl, alkynyl, aryl, aralkyl, heteroaryl, heteroaralkyl and acyl, respectively, as those terms are defined above. When any of the terms alkenyloxy, alkynyloxy, aryloxy, aralkyloxy and acyloxy is modified by “substituted,” it refers to the group —OR, in which R is substituted alkenyl, alkynyl, aryl, aralkyl, heteroaryl, heteroaralkyl and acyl, respectively.

The term “alkylamino” when used without the “substituted” modifier refers to the group —NHR, in which R is an alkyl, as that term is defined above. Non-limiting examples of alkylamino groups include: —NHCH₃, —NHCH₂CH₃, —NHCH₂CH₂CH₃, —NHCH(CH₃)₂, —NHCH(CH₂)₂, —NHCH₂CH₂CH₂CH₃, —NHCH(CH₃)CH₂CH₃, —NHCH₂CH(CH₃)₂, —NHC(CH₃)₃, —NH-cyclopentyl, and —NH-cyclohexyl. The term “substituted alkylamino” refers to the group —NHR, in which R is a substituted alkyl, as that term is defined above. For example, —NHCH₂CF₃ is a substituted alkylamino group.

The term “dialkylamino” when used without the “substituted” modifier refers to the group —NRR′, in which R and R′ can be the same or different alkyl groups, or R and R′ can be taken together to represent an alkanediyl having two or more saturated carbon atoms, at least two of which are attached to the nitrogen atom. Non-limiting examples of dialkylamino groups include: —NHC(CH₃)₃, —N(CH₃)CH₂CH₃, —N(CH₂CH₃)₂, N-pyrrolidinyl, and N-piperidinyl. The term “substituted dialkylamino” refers to the group —NRR′, in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached to the nitrogen atom.

The terms “alkoxyamino”, “alkenylamino”, “alkynylamino”, “arylamino”, “aralkylamino”, “heteroarylamino”, “heteroaralkylamino”, and “alkylsulfonylamino” when used without the “substituted” modifier, refers to groups, defined as —NHR, in which R is alkoxy, alkenyl, alkynyl, aryl, aralkyl, heteroaryl, heteroaralkyl and alkylsulfonyl, respectively, as those terms are defined above. A non-limiting example of an arylamino group is —NHC₆H₅. When any of the terms alkoxyamino, alkenylamino, alkynylamino, arylamino, aralkylamino, heteroarylamino, heteroaralkylamino and alkylsulfonylamino is modified by “substituted,” it refers to the group —NHR, in which R is substituted alkoxy, alkenyl, alkynyl, aryl, aralkyl, heteroaryl, heteroaralkyl and alkylsulfonyl, respectively.

The term “amido” (acylamino), when used without the “substituted” modifier, refers to the group —NHR, in which R is acyl, as that term is defined above. A non-limiting example of an acylamino group is —NHC(O)CH₃. When the term amido is used with the “substituted” modifier, it refers to groups, defined as —NHR, in which R is substituted acyl, as that term is defined above. The groups —NHC(O)OCH₃ and —NHC(O)NHCH₃ are non-limiting examples of substituted amido groups.

The term “alkylimino” when used without the “substituted” modifier refers to the group ═NR, wherein the alkylimino group is attached with one σ-bond and one π-bond, in which R is an alkyl, as that term is defined above. Non-limiting examples of alkylimino groups include: ═NCH₃, ═NCH₂CH₃ and ═N-cyclohexyl. The term “substituted alkylimino” refers to the group ═NR, wherein the alkylimino group is attached with one σ-bond and one π-bond, in which R is a substituted alkyl, as that term is defined above. For example, ═NCH₂CF₃ is a substituted alkylimino group.

Similarly, the terms “alkenylimino”, “alkynylimino”, “arylimino”, “aralkylimino”, “heteroarylimino”, “heteroaralkylimino” and “acylimino”, when used without the “substituted” modifier, refers to groups, defined as ═NR, wherein the alkylimino group is attached with one σ-bond and one π-bond, in which R is alkenyl, alkynyl, aryl, aralkyl, heteroaryl, heteroaralkyl and acyl, respectively, as those terms are defined above. When any of the terms alkenylimino, alkynylimino, arylimino, aralkylimino and acylimino is modified by “substituted,” it refers to the group ═NR, wherein the alkylimino group is attached with one σ-bond and one π-bond, in which R is substituted alkenyl, alkynyl, aryl, aralkyl, heteroaryl, heteroaralkyl and acyl, respectively.

The term “fluoroalkyl” when used without the “substituted” modifier refers to an alkyl, as that term is defined above, in which one or more fluorines have been substituted for hydrogens. The groups, —CH₂F, —CF₂H, —CF₃, and —CH₂CF₃ are non-limiting examples of fluoroalkyl groups. The term “substituted fluoroalkyl” refers to a non-aromatic monovalent group with a saturated carbon atom as the point of attachment, a linear or branched, cyclo, cyclic or acyclic structure, at least one fluorine atom, no carbon-carbon double or triple bonds, and at least one atom independently selected from the group consisting of N, O, Cl, Br, I, Si, P, and S. The following group is a non-limiting example of a substituted fluoroalkyl: —CFHOH.

The term “alkylphosphate” when used without the “substituted” modifier refers to the group —OP(O)(OH)(OR), in which R is an alkyl, as that term is defined above. Non-limiting examples of alkylphosphate groups include: —OP(O)(OH)(OMe) and —OP(O)(OH)(OEt). The term “substituted alkylphosphate” refers to the group —OP(O)(OH)(OR), in which R is a substituted alkyl, as that term is defined above.

The term “dialkylphosphate” when used without the “substituted” modifier refers to the group —OP(O)(OR)(OR′), in which R and R′ can be the same or different alkyl groups, or R and R′ can be taken together to represent an alkanediyl having two or more saturated carbon atoms, at least two of which are attached via the oxygen atoms to the phosphorus atom. Non-limiting examples of dialkylphosphate groups include: —OP(O)(OMe)₂, —OP(O)(OEt)(OMe) and —OP(O)(OEt)₂. The term “substituted dialkylphosphate” refers to the group —OP(O)(OR)(OR′), in which R and R′ can be the same or different substituted alkyl groups, one of R or R′ is an alkyl and the other is a substituted alkyl, or R and R′ can be taken together to represent a substituted alkanediyl with two or more saturated carbon atoms, at least two of which are attached via the oxygen atoms to the phosphorous.

The term “alkylthio” when used without the “substituted” modifier refers to the group —SR, in which R is an alkyl, as that term is defined above. Non-limiting examples of alkylthio groups include: —SCH₃, —SCH₂CH₃, —SCH₂CH₂CH₃, —SCH(CH₃)₂, —SCH(CH₂)₂, —S-cyclopentyl, and —S-cyclohexyl. The term “substituted alkylthio” refers to the group —SR, in which R is a substituted alkyl, as that term is defined above. For example, —SCH₂CF₃ is a substituted alkylthio group.

Similarly, the terms “alkenylthio”, “alkynylthio”, “arylthio”, “aralkylthio”, “heteroarylthio”, “heteroaralkylthio”, and “acylthio”, when used without the “substituted” modifier, refers to groups, defined as —SR, in which R is alkenyl, alkynyl, aryl, aralkyl, heteroaryl, heteroaralkyl and acyl, respectively, as those terms are defined above. When any of the terms alkenylthio, alkynylthio, arylthio, aralkylthio, heteroarylthio, heteroaralkylthio, and acylthio is modified by “substituted,” it refers to the group —SR, in which R is substituted alkenyl, alkynyl, aryl, aralkyl, heteroaryl, heteroaralkyl and acyl, respectively.

The term “thioacyl” when used without the “substituted” modifier refers to a monovalent group with a carbon atom of a thiocarbonyl group as the point of attachment, further having a linear or branched, cyclo, cyclic or acyclic structure, further having no additional atoms that are not carbon or hydrogen, beyond the sulfur atom of the carbonyl group. The groups, —CHS, —C(S)CH₃, —C(S)CH₂CH₃, —C(S)CH₂CH₂CH₃, —C(S)CH(CH₃)₂, —C(S)CH(CH₂)₂, —C(S)C₆H₅, —C(S)C₆H₄CH₃, —C(S)C₆H₄CH₂CH₃, —C(S)C₆H₃(CH₃)₂, and —C(S)CH₂C₆H₅, are non-limiting examples of thioacyl groups. The term “thioacyl” therefore encompasses, but is not limited to, groups sometimes referred to as “alkyl thiocarbonyl” and “aryl thiocarbonyl” groups. The term “substituted thioacyl” refers to a radical with a carbon atom as the point of attachment, the carbon atom being part of a thiocarbonyl group, further having a linear or branched, cyclo, cyclic or acyclic structure, further having at least one atom, in addition to the sulfur atom of the carbonyl group, independently selected from the group consisting of N, O, F, Cl, Br, I, Si, P, and S. The groups, —C(S)CH₂CF₃, —C(S)O₂H, —C(S)OCH₃, —C(S)OCH₂CH₃, —C(S)OCH₂CH₂CH₃, —C(S)OC₆H₅, —C(S)OCH(CH₃)₂, —C(S)OCH(CH₂)₂, —C(S)NH₂, and —C(S)NHCH₃, are non-limiting examples of substituted thioacyl groups. The term “substituted thioacyl” encompasses, but is not limited to, “heteroaryl thiocarbonyl” groups.

The term “alkylsulfonyl” when used without the “substituted” modifier refers to the group —S(O)₂R, in which R is an alkyl, as that term is defined above. Non-limiting examples of alkylsulfonyl groups include: —S(O)₂CH₃, —S(O)₂CH₂CH₃, —S(O)₂CH₂CH₂CH₃, —S(O)₂CH(CH₃)₂, —S(O)₂CH(CH₂)₂, —S(O)₂-cyclopentyl, and —S(O)₂-cyclohexyl. The term “substituted alkylsulfonyl” refers to the group —S(O)₂R, in which R is a substituted alkyl, as that term is defined above. For example, —S(O)₂CH₂CF₃ is a substituted alkylsulfonyl group.

Similarly, the terms “alkenylsulfonyl”, “alkynylsulfonyl”, “arylsulfonyl”, “aralkylsulfonyl”, “heteroarylsulfonyl”, and “heteroaralkylsulfonyl” when used without the “substituted” modifier, refers to groups, defined as —S(O)₂R, in which R is alkenyl, alkynyl, aryl, aralkyl, heteroaryl, and heteroaralkyl, respectively, as those terms are defined above. When any of the terms alkenylsulfonyl, alkynylsulfonyl, arylsulfonyl, aralkylsulfonyl, heteroarylsulfonyl, and heteroaralkylsulfonyl is modified by “substituted,” it refers to the group —S(O)₂R, in which R is substituted alkenyl, alkynyl, aryl, aralkyl, heteroaryl and heteroaralkyl, respectively.

The term “alkylsulfinyl” when used without the “substituted” modifier refers to the group —S(O)R, in which R is an alkyl, as that term is defined above. Non-limiting examples of alkylsulfinyl groups include: —S(O)CH₃, —S(O)CH₂CH₃, —S(O)CH₂CH₂CH₃, —S(O)CH(CH₃)₂, —S(O)CH(CH₂)₂, —S(O)-cyclopentyl, and —S(O)-cyclohexyl. The term “substituted alkylsulfinyl” refers to the group —S(O)R, in which R is a substituted alkyl, as that term is defined above. For example, —S(O)CH₂CF₃ is a substituted alkylsulfinyl group.

Similarly, the terms “alkenylsulfinyl”, “alkynylsulfinyl”, “arylsulfinyl”, “aralkylsulfinyl”, “heteroarylsulfinyl”, and “heteroaralkylsulfinyl” when used without the “substituted” modifier, refers to groups, defined as —S(O)R, in which R is alkenyl, alkynyl, aryl, aralkyl, heteroaryl, and heteroaralkyl, respectively, as those terms are defined above. When any of the terms alkenylsulfinyl, alkynylsulfinyl, arylsulfinyl, aralkylsulfinyl, heteroarylsulfinyl, and heteroaralkylsulfinyl is modified by “substituted,” it refers to the group —S(O)R, in which R is substituted alkenyl, alkynyl, aryl, aralkyl, heteroaryl and heteroaralkyl, respectively.

The term “alkylammonium” when used without the “substituted” modifier refers to a group, defined as —NH₂R⁺, —NHRR′⁺, or —NRR′R″⁺, in which R, R′ and R″ are the same or different alkyl groups, or any combination of two of R, R′ and R″ can be taken together to represent an alkanediyl. Non-limiting examples of alkylammonium cation groups include: —NH₂(CH₃)⁺, —NH₂(CH₂CH₃)⁺, —NH₂(CH₂CH₂CH₃)⁺, —NH(CH₃)₂⁺, —NH(CH₂CH₃)₂⁺, —NH(CH₂CH₂CH₃)₂⁺, —N(CH₃)₃⁺, —N(CH₃)(CH₂CH₃)₂⁺, —N(CH₃)₂(CH₂CH₃)⁺, —NH₂C(CH₃)₃⁺, —NH(cyclopentyl)₂⁺, and —NH₂(cyclohexyl)⁺. The term “substituted alkylammonium” refers —NH₂R⁺, —NHRR′⁺, or —NRR′R″⁺, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more carbon atoms, at least two of which are attached to the nitrogen atom shown in the formula.

The term “alkylsulfonium” when used without the “substituted” modifier refers to the group —SRR′⁺, in which R and R′ can be the same or different alkyl groups, or R and R′ can be taken together to represent an alkanediyl. Non-limiting examples of alkylsulfonium groups include: —SH(CH₃)⁺, —SH(CH₂CH₃)⁺, —SH(CH₂CH₂CH₃)⁺, —S(CH₃)₂⁺, —S(CH₂CH₃)₂⁺, —S(CH₂CH₂CH₃)₂⁺, —SH(cyclopentyl)⁺, and —SH(cyclohexyl)⁺. The term “substituted alkylsulfonium” refers to the group —SRR′⁺, in which R and R′ can be the same or different substituted alkyl groups, one of R or R′ is an alkyl and the other is a substituted alkyl, or R and R′ can be taken together to represent a substituted alkanediyl. For example, —SH(CH₂CF₃)⁺ is a substituted alkylsulfonium group.

The term “alkylsilyl” when used without the “substituted” modifier refers to a monovalent group, defined as —SiH₂R, —SiHRR′, or —SiRR′R″, in which R, R′ and R″ can be the same or different alkyl groups, or any combination of two of R, R′ and R″ can be taken together to represent an alkanediyl. The groups, —SiH₂CH₃, —SiH(CH₃)₂, —Si(CH₃)₃ and —Si(CH₃)₂C(CH₃)₃, are non-limiting examples of unsubstituted alkylsilyl groups. The term “substituted alkylsilyl” refers to —SiH₂R, —SiHRR′, or —SiRR′R″, in which at least one of R, R′ and R″ is a substituted alkyl or two of R, R′ and R″ can be taken together to represent a substituted alkanediyl. When more than one of R, R′ and R″ is a substituted alkyl, they can be the same of different. Any of R, R′ and R″ that are not either substituted alkyl or substituted alkanediyl, can be either alkyl, either the same or different, or can be taken together to represent a alkanediyl with two or more saturated carbon atoms, at least two of which are attached to the silicon atom.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of a compound of the present invention may be replaced by a sulfur or selenium atom(s).

A compound having a formula that is represented with a dashed bond is intended to include the formulae optionally having zero, one or more double bonds. Thus, for example, the structure

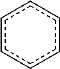

includes the structures

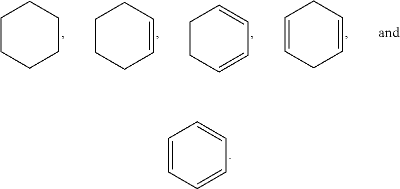

As will be understood by a person of skill in the art, no one such ring atom forms part of more than one double bond.

Any undefined valency on an atom of a structure shown in this application implicitly represents a hydrogen atom bonded to the atom.

As used herein, a “chiral auxiliary” refers to a removable chiral group that is capable of influencing the stereoselectivity of a reaction. Persons of skill in the art are familiar with such compounds, and many are commercially available.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained.

An “isomer” of a first compound is a separate compound in which each molecule contains the same constituent atoms as the first compound, but where the configuration of those atoms in three dimensions differs.

“Optional” or “optionally” means that the subsequently described event or circumstance may or may not occur, and that the description includes instances where said event or circumstance occurs and instances in which it does not. It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. “Optionally substituted” refers to all subsequent modifiers in a term, for example in the term “optionally substituted C₁₋₈alkylaryl,” optional substitution may occur on both the “C₁₋₈alkyl” portion and the “aryl” portion of the molecule; and for example, optionally substituted alkyl includes optionally substituted cycloalkyl groups, which in turn are defined as including optionally substituted akyl groups, potentially ad infinitum. If a hetercyclic ring is “optionally substituted,” then both the carbon and any heteroatoms in the ring may be substituted thereon. Examples of optional substitution include, but are not limited to alkyl, halogen, alkoxy, hydroxy, oxo, carbamyl, acylamino, sulfonamido, carboxy, alkoxycarbonyl, acyl, alkylthio, alkylsulfonyl, nitro, cyano, amino, alkylamino, cycloalkyl and the like. Thus, for example, if a group “—C(O)R” is described, where “R” is optionally substituted alkyl, then, “R” would include, but not be limited to, —CH₂Ph, —CH₂CH₂OPh, —CH═CHPhCH₃, —C₃H₄CH₂N(H)Ph, and the like.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

As used herein, “predominantly one enantiomer” means that a compound contains at least about 85% of one enantiomer, or more preferably at least about 90% of one enantiomer, or even more preferably at least about 95% of one enantiomer, or most preferably at least about 99% of one enantiomer. Similarly, the phrase “substantially free from other optical isomers” means that the composition contains at most about 15% of another enantiomer or diastereomer, more preferably at most about 10% of another enantiomer or diastereomer, even more preferably at most about 5% of another enantiomer or diastereomer, and most preferably at most about 1% of another enantiomer or diastereomer.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “repeat unit” is the simplest structural entity of certain materials, for example, frameworks and/or polymers, whether organic, inorganic or metal-organic. In the case of a polymer chain, repeat units are linked together successively along the chain, like the beads of a necklace. For example, in polyethylene, —[—CH₂CH₂—]ₙ—, the repeat unit is —CH₂CH₂—. The subscript “n” denotes the degree of polymerisation, that is, the number of repeat units linked together. When the value for “n” is left undefined, it simply designates repetition of the formula within the brackets as well as the polymeric nature of the material. The concept of a repeat unit applies equally to where the connectivity between the repeat units extends three dimensionally, such as in metal organic frameworks, cross-linked polymers, thermosetting polymers, etc.

The term “saturated” when referring to an atom means that the atom is connected to other atoms only by means of single bonds.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are minor images of each other, like left and right hands. “Diastereomers” are stereoisomers of a given compound that are not enantiomers.

The invention contemplates that for any stereocenter or axis of chirality for which stereochemistry has not been defined, that stereocenter or axis of chirality can be present in its R form, S form, or as a mixture of the R and S forms, including racemic and non-racemic mixtures.

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Effective amount,” “Therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other abbreviations used herein are as follows: DMSO, dimethyl sulfoxide; NO, nitric oxide; iNOS, inducible nitric oxide synthase; COX-2, cyclooxygenase-2; NGF, nerve growth factor; IBMX, isobutylmethylxanthine; FBS, fetal bovine serum; GPDH, glycerol 3-phosphate dehydrogenase; RXR, retinoid X receptor; TGF-β, transforming growth factor-β; IFNγ or IFN-γ, interferon-γ; LPS, bacterial endotoxic lipopolysaccharide; TNFα or TNF-α, tumor necrosis factor-α; IL-1β, interleukin-1β; GAPDH, glyceraldehyde-3-phosphate dehydrogenase; MTT, 3-[4,5-dimethylthiazol-2-yl]-2,5-diphenyltetrazolium bromide; TCA, trichloroacetic acid; HO-1, inducible heme oxygenase.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### III. Pharmaceutical Preparations

Pharmaceutical compositions of the present invention comprise an effective amount of one or more antimicrobial compositions dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one antimicrobial composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The antimicrobial composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The antimicrobial composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the antimicrobial composition, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the antimicrobial composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the antimicrobial composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, the antimicrobial composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound antimicrobial composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Kits of the Invention

Any of the compositions described herein may be comprised in a kit. The kits will thus comprise, in suitable container means, an antimicrobial composition of the present invention. In some embodiments, the kit further comprises an additional agent for treating a microbial infection, and the additional agent may be combined with the composition of the invention or may be provided separately in the kit. In some embodiments, means of taking a sample from an individual and/or of assaying the sample may be provided in the kit. In certain embodiments for kits related to malaria infection, there may be means to identify malaria infection from an individual, such as Giemsa stain, diagnostic antibodies, or PCR primers and reagents, for example.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the antimicrobial composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following examples are offered by way of example and are not intended to limit the scope of the invention in any manner.

### Example 1

### Design and Synthesis of Pyridine-containing Inhibitors of 1-Deoxy-d-Xylulose-5-Phosphate Reductoisomerase

Eukaryotic parasite Plasmodium spp. are the causative agents of malaria, among which Plasmodium falciparum produces the most severe form of human malaria and is responsible for the vast majority of deaths of malaria patients. Every year, approximately 300-500 million people are afflicted by malaria and >1 million die of the disease with most being children under the age of five. In addition, these dreadful numbers could be rising because of the increasing drug resistance of P. falciparum against inexpensive drugs such as chloroquine. Since 2001, the World Health Organization has strongly recommended to use artemisinin based combination therapies to treat malaria. However, Plasmodium parasites are known to be able to quickly develop drug resistance. After 10 years, P. falciparum strains that are resistant to these new drug combinations have started to appear. There is therefore a pressing need to develop new antimalarial drugs.

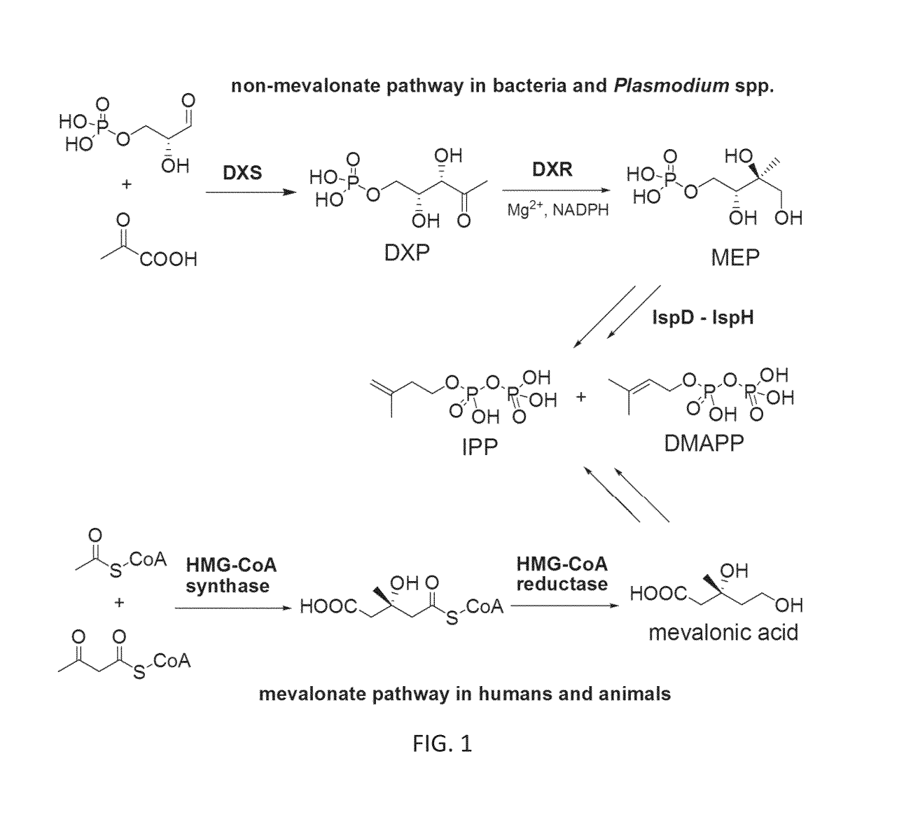

1-Deoxy-D-xylulose-5-phosphate reductoisomerase (DXR) is the second enzyme in the non-mevalonate isoprene biosynthesis pathway (FIG. 1), catalyzing the reductive isomerization of 1-deoxy-D-xylulose-5-phosphate (DXP) to 2-methyl-D-erythritol-4-phosphate (MEP) using Mg2+ (or Mn2+) and NADPH as the cofactors. This is used by most bacteria as well as apicomplexan parasites such as P. falciparum, to make essential isopentenyl diphosphate (IPP) and dimethylallyl diphosphate (DMAPP), which are the only two precursors for biosynthesis of all isoprenoids/terpenoids, including important substances such as isoprenyl (e.g., farnesyl and undecaprenyl) diphosphates, vitamins and steroids. DXR is essential for the growth of all these species including P. falciparum, while humans and animals use the mevalonate pathway to synthesize IPP and DMAPP, making DXR an attractive target for finding novel antimalarial drugs.

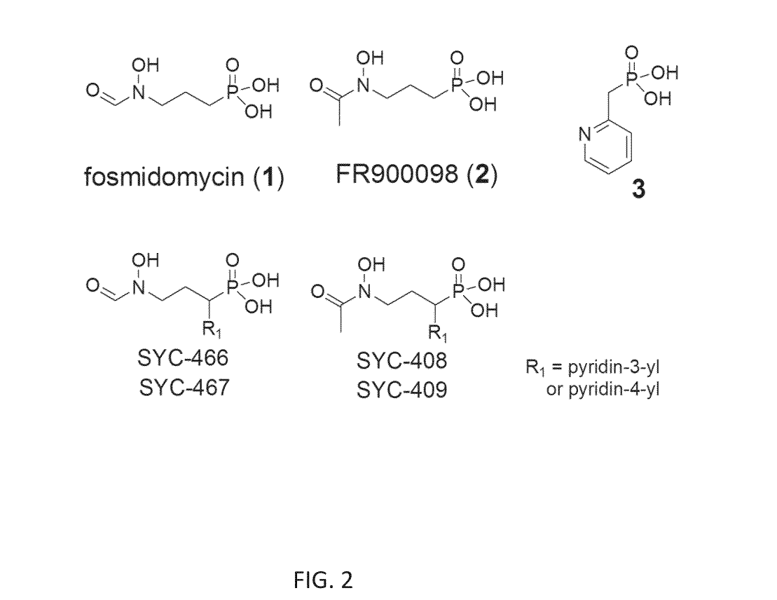

Fosmidomycin (1, FIG. 2) and its close analog FR900098 (2) were found to be potent DXR inhibitors and possess antibacterial and malarial activity. Particularly, several recent clinical trials showed 1 (in combination with clindamycin) is safe for human use and effective against P. falciparum malaria. However, due to very short half life in human plasma (˜1 h) as well as poor oral availability of 1, considerable interest has therefore generated to develop potent, lipophilic DXR inhibitors with the rationale that these compounds could have broad anti-infective activities with improved pharmacokinetic properties.

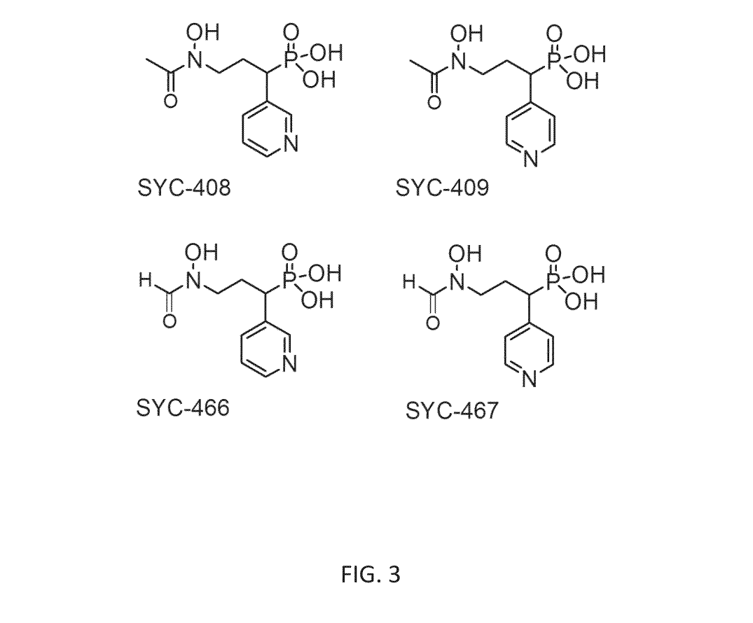

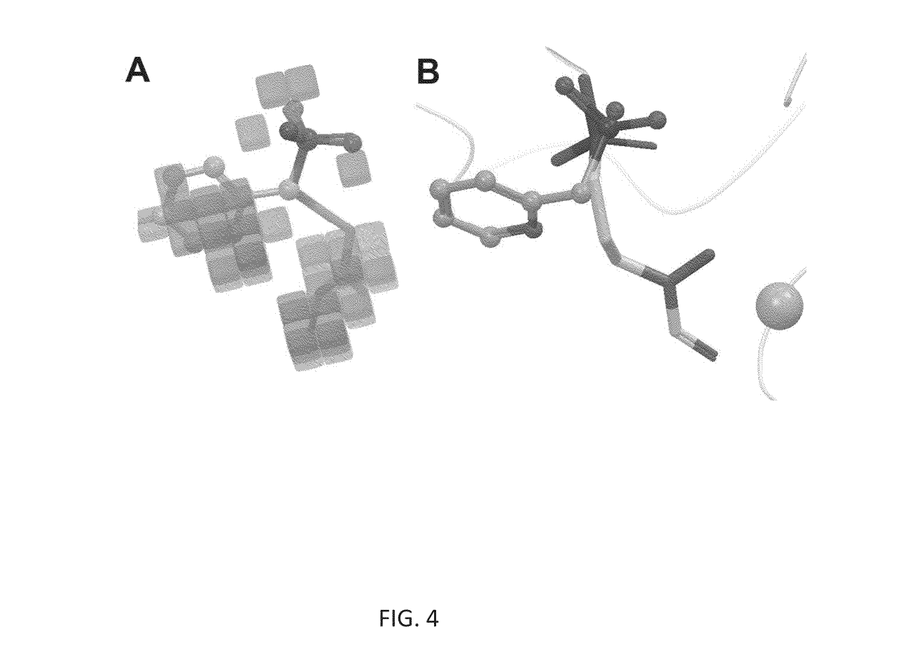

A series of lipophilic, pyridine- or quinoline-containing phosphonate compounds, such as compound 3 (FIG. 2), were found to be a new class of DXR inhibitors. Structure activity relationship (SAR) and quantitative SAR (QSAR) studies all show the importance of the presence of an electron-deficient aromatic ring, such as a pyridine, at the α-position of the phosphonate group (FIG. 4A). In addition to this, superposition of the crystal structures of E. coli DXR (EcDXR) in complex with 1 and 3 (FIG. 4B) indicates fosmidomycin derivatives with an α-pyridine substituent, such as compounds SYC-466, -467, -408, -409 shown in FIGS. 2 and 3, in certain embodiments are novel DXR inhibitors with improved potency, since they could possess favorable interactions with DXR found in both 1 and 3.

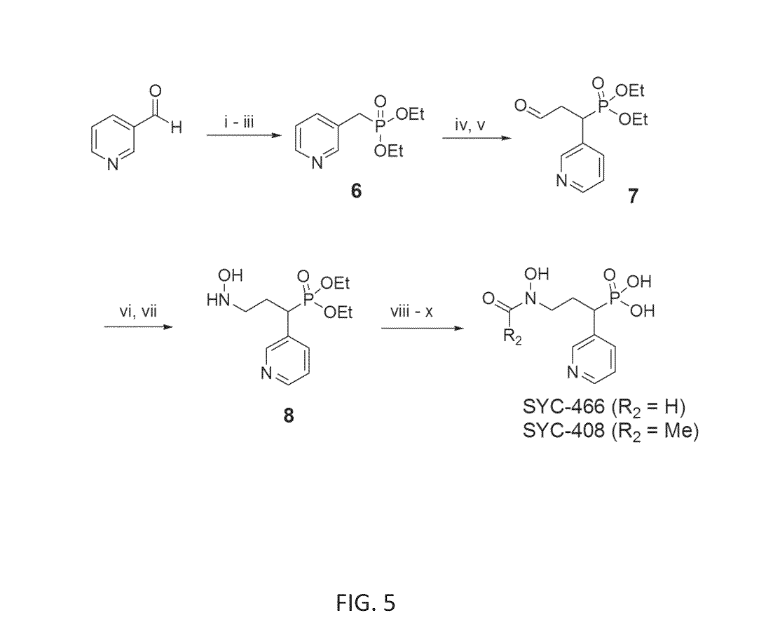

The general method for synthesizing compounds SYC-466, -467, -408, -409 is shown in FIG. 5. Pyridine-3-carboxaldehyde was reduced and converted to 3-picolinyl chloride, which was reacted with sodium salt of diethylphosphite to give compound 6. It was alkylated with allyl bromide, followed by ozonization to give aldehyde 7. Upon reductive amination using O-benzyl-hydroxylamine and NaCNBH₃, the resulting compound 8 was formylated or acetylated, followed by selective hydrolysis using bromo-trimethylsilane (TMSBr) and hydrogenation, affording compounds SYC-466, -408. Compounds SYC-467, -409 can be similarly prepared, starting from pyridine-4-carboxaldehyde. However, analogous compounds with a pyridin-2-yl substituent cannot be synthesized from pyridine-2-carboxaldehyde, due to instability of the corresponding aldehyde 7 (or its O-benzyl-hydroxylamine oxime). ^{a}Reagents and conditions: (i) NaBH₄, MeOH; (ii) SOCl₂; (iii) HP(O)(OEt)₂, NaH, THF (iv) n-BuLi, then allyl bromide, −78-0° C.; (v) O₃, −78° C., then Me₂S; (vi) NH₂OBn; (vii) NaCNBH₃, pH 3, MeOH (viii) For R₂═H, HCOOH/Ac₂O; for R₂=Me, Ac₂O; (ix) TMSBr; (x) H₂, Pd/C.

### Example 2

### Enzyme Inhibition of Pyridine-Containing Inhibitors of 1-Deoxy-d-Xylulose-5′-Phosphate Reductase

The enzyme activities of the newly synthesized compounds SYC-466, -467, -408, -409 were first tested against recombinant EcDXR. As shown in Table 1, compounds SYC-467, -408 were found to exhibit potent activity with Kᵢ values of 35 and 42 nM, respectively, being as active as 1 (Kᵢ=34 nM). Compounds SYC-466, -409 (Kᵢ=87 and 82 nM) have, however, slightly reduced inhibition against EcDXR. The inventors next tested compounds SYC-466, -467, -408, -409 against recombinant P. falciparum DXR (PfDXR). It is noted that EcDXR has been used in the vast majority of previous studies, despite EcDXR inhibition may not be predictive in the context of developing antimalarial drugs.

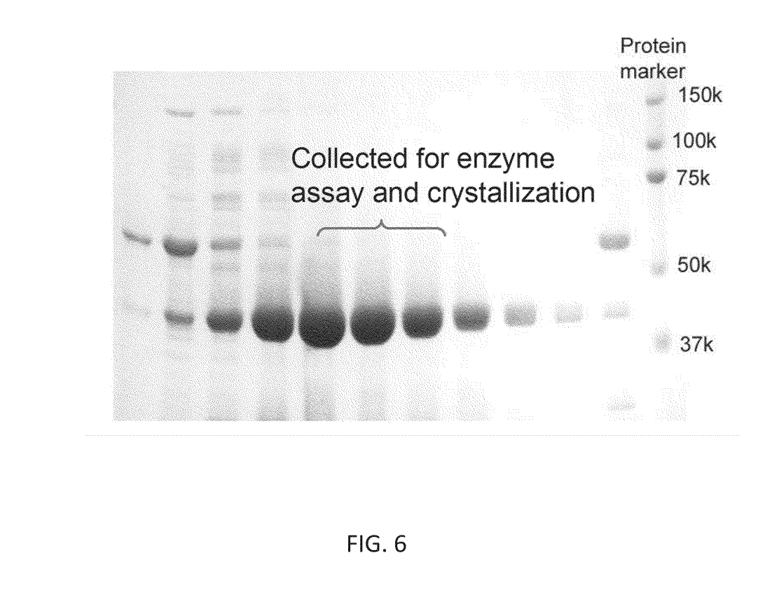

PfDXR (catalytic domain 75-488) was cloned from P. falciparum genomic DNA using 5′-GCGGATCCAAGAAACCAATTAATGTAGC-3′ (SEQ ID NO:1) and 5′-GCAAGCTTCTATGAAGAATTATGTTTGTTGT-3′ (SEQ ID NO:2) as forward and reverse primers, respectively, and was inserted into pQE30 expression vector (Qiagen). The correctness of insert was verified by sequencing. The plasmid was transformed into E. coli (M15 strain) and cultured in LB medium containing kanamycin (25 μg/mL) and ampicillin (50 μg/mL). Upon reaching an optical density of ˜0.6 at 600 nm, PfDXR expression was induced by adding 0.2 mM isopropylthiogalactoside (IPTG) for 5 hours at 37° C. Cells were harvested and disrupted and His6-tagged recombinant PfDXR was purified using a standard protocol (Ni-affinity followed by Superdex 75 column chromatography). PfDXR was obtained with >90% purity (FIG. 6) and found to be enzymatically active, using 50 nM enzyme, 100 μM DXP (substrate), 100 μM NADPH (cofactor), 1 mM MnCl₂ (cofactor) in 50 mM HEPES buffer (pH 7.6) containing 50 μg/mL BSA. The reaction can be monitored, same as those for E. coli and M. tuberculosis DXRs, with a decreasing absorbance at 340 nm (due to NADPH consumption) using a Beckman DTX-880 microplate reader. PfDXR kinetic studies determined the Kₘ value for the substrate DXP is 106 μM, which is similar to that of EcDXR and would be used to calculate the Kᵢ values of inhibitors.

Consistent with a previous report, fosmidomycin (1) was found to be also a very potent inhibitor of PfDXR with a Kᵢ value of 21 nM. However, DXR inhibitors SYC-466, -467, -408, -409 possess considerably higher activity against PfDXR (Table 1). The Kᵢ values of these compounds range from 1.9 to 13 nM, with the best compound 5a (Kᵢ=1.9 nM) being ˜10-fold more active than fosmidomycin.

TABLE 1
Enzyme inhibition constants (Ki) and P. falciparum proliferation
inhibitory activity (IC50).

IC₅₀ against P.
falciparum
proliferation (μM)

| | | |
|---|---|---|
| Enzyme Kᵢ (μM) | 3D7 | Dd2 |

| | EcDXR | PfDXR | strain | strain |
|---|---|---|---|---|
| Fosmidomycin | 0.034 | 0.021 | 1.17 | 0.44 |
| (1) | | | | |
| SYC-466 | 0.087 | 0.013 | 0.34 | 0.18 |
| SYC-467 | 0.035 | 0.0089 | 0.18 | 0.17 |
| SYC-408 | 0.042 | 0.0019 | 0.44 | 0.31 |
| SYC-409 | 0.082 | 0.013 | 0.63 | 0.46 |
| chloroquinone | NT | NT | 0.028 | 0.111 |

### Example 3

### Antimalarial Activity of Pyridine-Containing Inhibitors of 1-Deoxy-D-Xylulose-5-Phosphate Reductoisomerase

It was next tested if compounds SYC-466, -467, -408, -409 have antimalarial activity against in vitro cultured P. falciparum. A fluorescence based protocol (43) using 4′,6-diamidino-2-phenylindole (DAPI) as a DNA stain was used. P. falciparum strains 3D7 (chloroquine sensitive) and Dd2 (multidrug resistant, including chloroquine) were acquired from MR4 (Manassas, Va.). P. falciparum was cultured in type O human erythrocytes (Zen-Bio Inc, NC) in a RPMI 1640 medium supplemented with 10% human serum, 25 μg/mL gentamicin, 0.006% HEPES and 0.002% NaHCO₃ (pH 7.2) in a gas environment of 5% CO₂, 5% O₂, and 90% N₂ at 37° C. Upon synchronizing the parasite with 5% sorbitol at the ring stage, P. falciparum (0.5% parasitemia and 2% hematocrit, 200 μL/well) in 96-well plates was treated with different concentrations of chloroquine, fosmidomycin and compounds SYC-466, -467, -408, -409 (3 nM to 30 μM) for 3 days. Upon centrifugation followed by careful removal of the medium, a mixture (100 μL/well) containing DAPI (5×10⁻⁵ mg/mL), 20 mM Tris, 5 mM EDTA, 0.008% saponin and 0.001% Triton X-100 was added, incubated for 30 min, and fluorescence of each well determined using a DTX-880 microplate reader (excitation/emission at 360/460 nm). Data were processed using a standard sigmoidal dose response fitting in Prism 5.0 to generate EC₅₀ values.

Antimalarial activities of the pyridine-containing DXR inhibitors SYC-466, -467, -408, -409 were evaluated against the growth of two strains of erythrocyte-stage P. falciparum. The 3D7 strain is drug-sensitive, while the Dd2 strain is multi-drug resistant, including chloroquine (CQ), pyrimethamine and mefloquine. Chloroquine and fosmidomycin were used as two positive controls. As can be found in Table 1, these compounds especially SYC-466 and -467 have potent antimalarial activity, more active than fosmidomycin. In addition, their activity is not compromised when treating chloroquine resistant Dd2 strain of P. falciparum.

### Example 4

### Cytotoxicity of DXR Inhibitors

PfDXR inhibitors SYC-466, -467, -408, -409 were tested against three non-cancerous human cell lines, i.e., WI-38 (fibroblast), HEK293 (kidney) and Beas2B (lung epithelial), to evaluate the potential toxicity of these compounds, using an assay routinely perform (41). 1×10⁵ cells are inoculated into each well of a 96-well plate and cultured in Dulbecco's Modified Eagle's Medium (DMEM) supplemented with 10% fetal bovine serum at 37° C. in a 5% CO₂ atmosphere with 100% humidity overnight for cell attachment. After addition of compounds (from 0.1-300 μM), plates are incubated for 48 h after which cell viability are assessed by the [3-(4,5-dimethylthiazol-2-yl)-5-(3-carboxymethoxyphenyl)-2-(4-sulfophenyl)-2H-tetrazolium, inner salt (MTS) assay, using a commercially available kit (Promega). IC₅₀s of each compound can be calculated from dose response curves.

PfDXR inhibitors SYC-466, -467, -408, -409 were found to exhibit essentially no cytotoxicity even at 300 μM against the growth of human non-cancerous fibroblast cells (WI-38) as well as two tumor cell lines (Hela and A549).

### Example 5

### Exemplary Research Design and Methods

Medicinal Chemistry and SAR Studies

The overall objective of this embodiment is to use rational drug design and organic chemistry to synthesize novel PfDXR inhibitors. The rationale is that because novel compounds SYC-466, -467, -408, -409 show antimalarial activity, lipophilic PfDXR inhibitors that possess more potent in vitro and in vivo activity are needed.

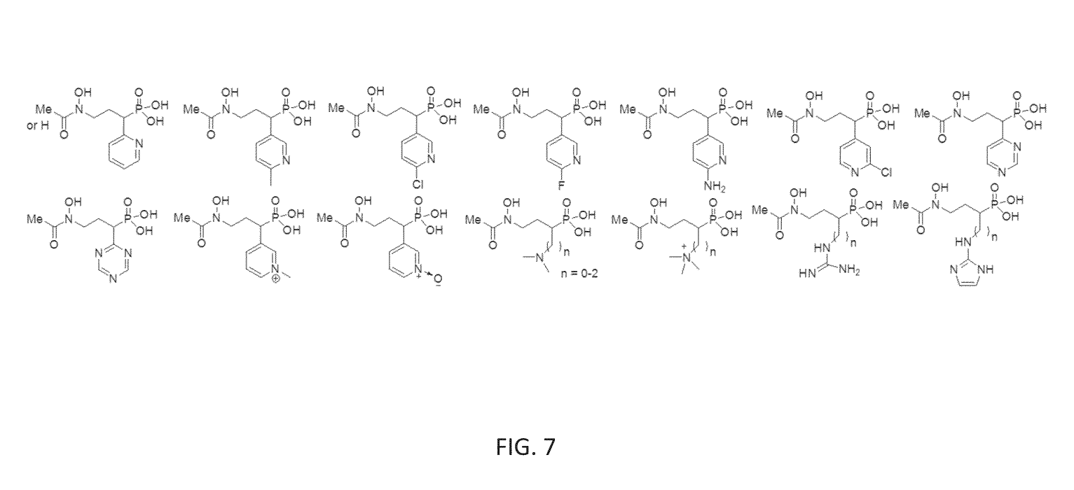

Design and synthesis of analogs of compounds (SYC-466, -467, -408, -409). Based on the rational, structure-based design, compounds 7-10 have been found to be very potent inhibitors (Kᵢ: 1.9-13 nM) of P. falciparum DXR (PfDXR). The high potency could be attributed to the strong π-π stacking/charge transfer interactions between their electron-deficient pyridine ring and the electron-rich indole group of the conserved Trp residue (Trp296 of PfDXR). One can use medicinal chemistry to synthesize the exemplary compounds that have DXR activity, including improved activity (see FIG. 7)

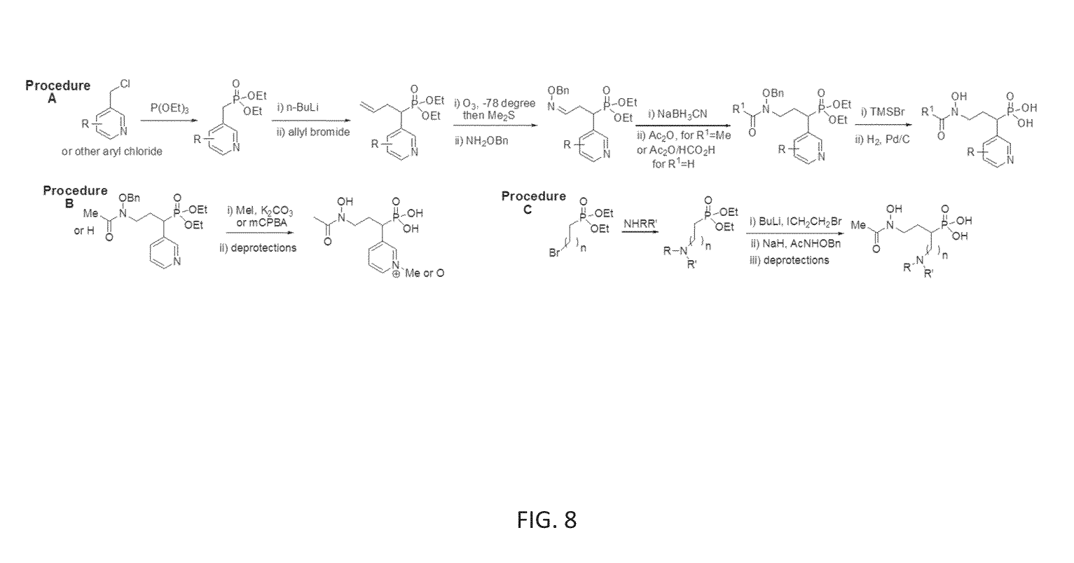

A common feature of these compounds is that they all contain a highly electron-deficient aromatic or a positively charged group (at the physiological pH), including pyridine, pyrimidine, pyridinium, tertiary amine, quaternary ammonium, guanidine and aminoimidazole. These groups are well-known to have strong π-π stacking and/or cation-π interactions (44-47) with electron-rich tryptophan residue in proteins. Consequently, potent PfDXR inhibitors are expected to be found among these compounds. The general procedures A-C in FIG. 8 may be used to synthesize compounds of the invention.

Quantitative structure activity relationship (QSAR) studies. After obtaining at least >30 DXR inhibitors as well as their biological activity data, one can use the program Phase in Schrodinger Suite (version: 2011) to perform 3-dimensional QSAR studies, which was shown previously (22) to be an effective method to guide rational inhibitor design. All compounds can be built and geometry- and energy-minimized using the OPLS-2005 force field in the program Maestro and their 3-D structures thus obtained be aligned using the “Flexible Ligand Alignment” module in Maestro. The aligned compounds can be imported into the Phase program. A partial least-squares (PLS) method can be used to correlate the activities of these compounds with the 3-D structural fields calculated by Phase with default settings. If a good QSAR model with R² of >0.85 and Q² (cross-validated R²) of >0.5 is obtained, it can then be further validated by performing at least 5 leave-n-out (n≧3) training/test sets, in order to find if the model has satisfactory predictivity. With these QSAR results, in which compound activities are correlated to their 3-D structures in a quantitative manner, one can optimize the electrostatic, steric, hydrophobic and H-donor/acceptor field requirements for PfDXR inhibition and therefore guide further inhibitor design and synthesis. Moreover, a potent enzyme inhibitor may not always have good cell activity (48) and there can be a poor correlation between the enzyme and cell activity, because cell membrane permeability as well as other factors impact the effectiveness of the inhibitor. For example, compound SYC-408 is several times more active than compound 4b against PfDXR enzyme (Table 1). However, SYC-408was found to be several times less active in killing P. falciparum (both 3D7 and Dd2 strains) than SYC-467. To more accurately correlate and/or predict these two sets (enzyme and cell) of activity data, QSAR methods can be used to regress the cell activity with the enzyme activity in combination with other molecular descriptors (e.g., logP value) calculated by the program Qprop in Schrodinger. Since Plasmodium killing activity is more relevant to drug discovery, QSAR models generated here can complement 3D QSAR based inhibitor design.

### Example 6

### Expression, Characterization and Inhibition of Toxoplasma gondii 1-Deoxy-d-Xylulose-5-Phosphate Reductoisomerase

The unicellular protozoan parasite Toxoplasma gondii, the causative agent of toxoplasmosis, is an important human pathogen (Montoya and Liesenfeld, 2004). In healthy adults, toxoplasmosis typically only produces mild, flu-like symptoms and the parasite becomes dormant. However, three factors make T. gondii a threat to public health. First, the parasite is highly promiscuous, infecting almost all warm-blooded animals including humans, with cats being the definitive host. Humans are infected by contacting cat feces contaminated with the mature oocyst form or by consumption of undercooked meat carrying tissue cysts. It is estimated that ˜30% of world population is chronically infected with T. gondii. A recent CDC (Centers for Disease Control and Prevention) report disclosed that the prevalence of this infecction in the US is ˜11% (Montoya and Liesenfeld, 2004; Jones et al,. 2007). Second, approximately one third of women infected for the first time with T. gondii during pregnancy will pass the parasite to the fetus where it can cause serious neurological damage to the fetus. Infection in particular the first trimester can lead to stillbirth. Third, the parasite poses a significant threat to immunocompromised persons, such as HIV-AIDS, cancer or organ transplant patients. Under these conditions latent infection can reactivate to fulminant Toxoplasma encephalitis, a life-threatening condition. Immunocompromised patients therefore may require recurrent treatment as current treatments are unable to clear the chronic infection. This is also true for immunocompetent patients suffering from recurring ocular toxoplasmosis. Current therapy is largely limited to anti-folate therapy. Long-term use of sulfonamides in particular has significant side effects including hypersensitivity. New therapeutic agents are therefore needed to treat toxoplasmosis.

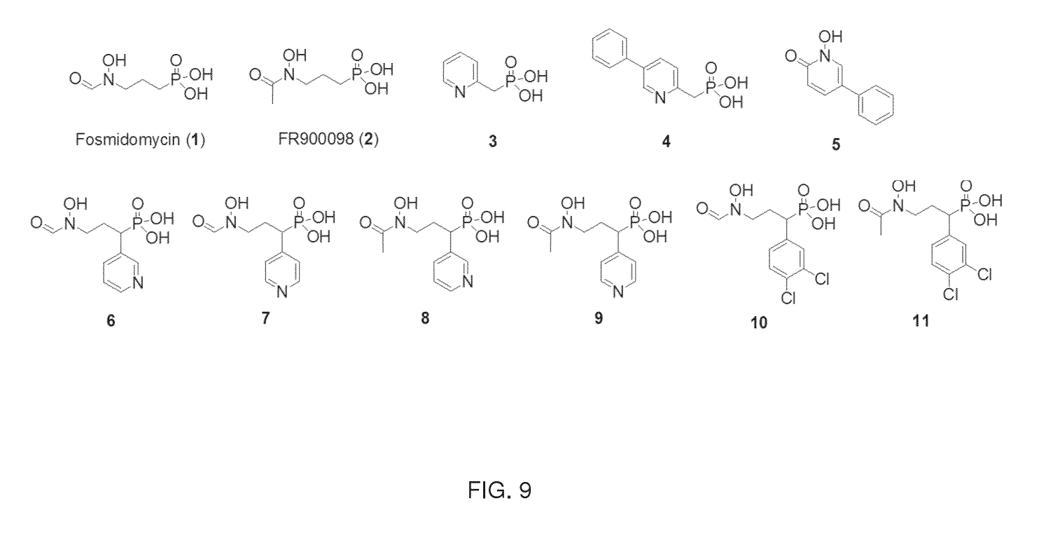

1-Deoxy-D-xylulose-5-phosphate reductoisomerase (DXR) in the MEP (2-C-methyl-D-erythritol-4-phosphate) isoprene biosynthesis pathway is a novel target for developing anti-infective drugs (Hunter, 2007; Singh et al.,2007; Obiol-Pardo et al., 2011). As shown in FIG. 1, unlike humans and animals that use the mevalonate pathway, most bacteria and apicomplexan parasites, including T. gondii and Plasmodium spp. (malaria parasites), use exclusively the MEP pathway to synthesize isopentenyl diphosphate (IPP) and its isomer dimethylallyl diphosphate (DMAPP), essential intermediates for the synthesis of isoprenoid compounds. DXR is the 2^{nd} enzyme of the pathway, catalyzing the reduction and isomerization of 1-deoxy-D-xylulose-5-phosphate (DXP) to 2-C-methyl-D-erythritol-4-phosphate (MEP) using Mg²⁺ and NADPH as enzyme cofactors (FIG. 1). Fosmidomycin (FIGS. 2 and 9), a naturally occurring antibiotic, has been found to be a potent inhibitor of DXR⁶ and possess antibacterial and antimalarial activities in preclinical studies and clinical trials (Mine et al., 1980; Jomaa et al., 1999; Missinou et al., 2002; Oyakhirome et al., 2007). This has further validated DXR as a promising drug target. Due to the poor pharmacokinetics of fosmidomycin (very short half-life in plasma and low oral availability), there is considerable interest in finding more potent and stable DXR inhibitors (Deng et al., 2009; Deng et al., 2011; Xue et al., DOI:10.1021/m1300419r; Cai et al., 2012; Haimers et al., 2006; Silber et al., 2005; Kuntz et al., 2005; Merckle et al., 2005; Munos et al., 2008; Ortmann et al., 2007). Medicinal chemistry studies by us and other groups have resulted in the synthesis of structurally diverse DXR inhibitors and representative examples are shown in FIG. 9. None of these DXR inhibitors showed activity against T. gondii growth. This was surprising considering the finding that T. gondii DXR (TgDXR) is essential to the growth of this organism (Nair et al., 2011). T. gondii resistance to fosmidomycin is due to limited drug uptake, as previously found for certain bacteria (Dhiman et al., 2005; Brown and Parish, 2008). The parasite cell membrane represents a permeability barrier for the compound. This is supported by the observation that fosmidomycin can effectively kill a strain of T. gondii engineered to express the bacterial GlpT, a known transporter of fosmidomycin, thus validating TgDXR as a target for developing novel anti-toxoplasmosis drugs (Nair et al., 2011). In the present example, the inventors demonstrate the expression, purification and biochemical characterization of recombinant T. gondii DXR (TgDXR). The inhibitory activity as well as structure activity relationships of TgDXR inhibitors are also provided.

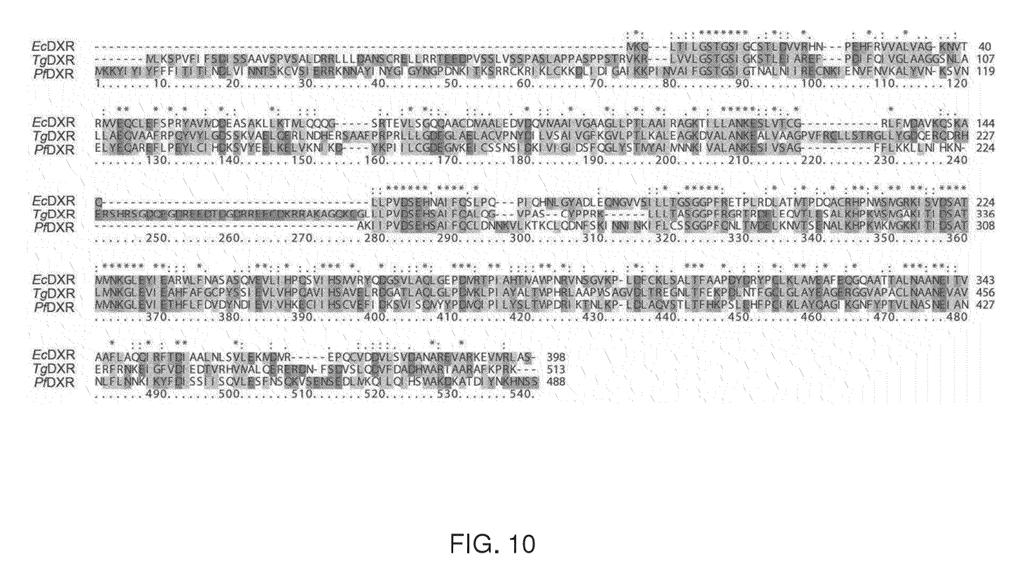

First, the inventors performed multiple protein alignments of the putative TgDXR (NCBI Reference Sequence: XP_{—}002370806.1) with E. coli (Ec) and P. falciparum (Pf) DXRs, and the result is shown in FIG. 10. Similar to PfDXR, TgDXR was found to carry an additional 67 amino acid residue extension at the N-terminal, when compared with the E. coli enzyme. This sequence in specific embodiments represents the bipartite apicoplast targeting peptide (Jomaa et al., 1999), because both proteins localize to the apicoplast of the parasites. In addition, TgDXR possesses a very long linking sequence (224-285) with 62 residues between the NADPH binding domain (68-223) and the metal/substrate binding domain (286-513). However, in EcDXR and PfDXR, no more than 13 amino acid residues, which are mostly located in an α-helix that is away from the enzyme's active site, link the two domains. Nevertheless, the low homology among these three linker peptides (FIG. 10) as well as the structural information from EcDXR and PfDXR indicate the segment 224-285 of TgDXR may not be important for enzyme activity. Except for these differences, these three enzymes share an overall high degree of similarity.

The inventors next cloned the catalytic domain (68-513) of TgDXR and inserted it into the expression plasmid pET24b. The plasmid was transformed into E. coli BL21-CodonPlus strain and cultured in LB medium containing kanamycin and chloramphenicol (The plasmid was transformed into E. coli (BL21-CodonPlus strain from Agilent) and cultured in LB medium containing kanamycin (25 μg/mL) and chloramphenicol (34 μg/mL). Upon reaching an optical density of ˜0.6 at 600 nm, TgDXR expression was induced by adding 0.25 mM isopropylthiogalactoside (IPTG) for 4 hours at 37° C. Cells were then harvested and resuspended in 50 mM NaH₂PO₄ (pH 8.0), 300 mM NaCl (buffer A) containing 20 mM imidazole. After addition of 0.2 mM phenylmethylsulfonyl fluoride and sonication at 0° C., the lysate was centrifuged at 20,000 rpm for 25 min and the supernatant was collected and subjected to an affinity column chromatography using the Ni-affinity column chromatography (HiTrap IMAC FF from GE Healthcare). The resin was washed with 30 mM imidazole in buffer A and then the protein was eluted with 300 mM imidazole in buffer A. After desalting (HiTrap Desalting, GE Healthcare) to 20 mM Tris pH 7.5, 150 mM NaCl, 2% glycerol, the protein was concentrated and stored in small aliquots at ˜80° C.).

His6-tagged recombinant TgDXR was expressed and purified using a standard Ni-affinity column chromatography to ˜90% purity, showing an apparent molecular mass of ˜45 kD.

The recombinant enzyme was biochemically characterized and found to be able to catalyze the conversion of DXP to MEP in the presence of Mg²⁺ and NADPH (The enzyme activity was determined in 96-well microplates using purified TgDXR (100 nM), 4 mM MgCl₂, 100 μM DXP, 100 μM NADPH in 50 mM HEPES buffer (pH=7.6) containing 50 μg/mL bovine serum albumin (BSA). For inhibition assays, compounds were incubated with TgDXR for 10 min at 30° C. before adding DXP to initiate the reaction. The reaction rate was monitored at 340 nm using a Beckman DTX-880 microplate reader. The initial velocities of wells containing increasing concentrations of an inhibitor were calculated and imported into Prism (version 5.0, GraphPad Software, Inc., La Jolla, Calif.). The IC₅₀ values were then obtained by using a standard dose response curve fitting. For less potent inhibitors (IC₅₀>500 nM), K, values were calculated using the formula Kᵢ=IC₅₀/(1+[S]/Kₘ), where [S] is the concentration of DXP (100 μM) and Kₘ was determined to be 25.5 μM. For highly potent inhibitors (IC₅₀≦500 nM), the Morrison tight inhibition equation in Prism was used to calculate their Kᵢ values.).

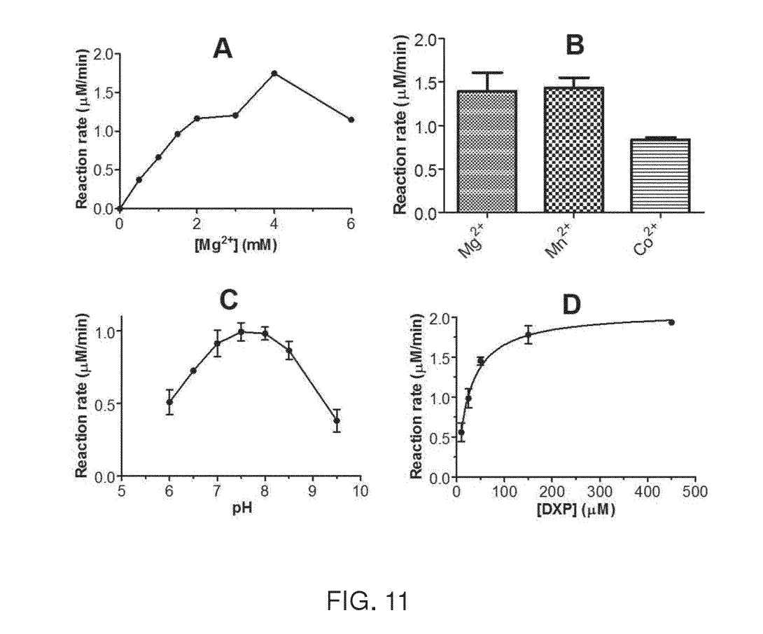

The reaction rate was monitored at 340 nm, where NADPH UV absorbance is maximal. First, the activity was tested in a HEPES buffer (50 mM, pH=7.6) containing TgDXR (100 nM), DXP (100 μM), NADPH (100 μM), 50 μg/mL BSA (bovine serum albumin) and varying concentrations of MgCl₂. As shown in FIG. 11a, the activity of TgDXR is dependent on Mg²⁺, the enzyme is completely inactive in the absence Mg²⁺ and activity increases with higher [Mg²⁺] until reaching a maximum at 4 mM Mg²⁺. Activity of the enzyme can also be supported by Mn²⁺ and Co²⁺, two additional commonly used divalent metal ions, as illustrated in FIG. 11b. In the presence of Mn²⁺ (2 mM) TgDXR exhibits essentially the same activity as with Mg²⁺, and shows approximately half of the activity with Co²⁺ (2 mM). In addition, the inventors measured the pH-dependence of TgDXR and the results demonstrated a pH optimum of 7.5-8.0 for this enzyme (FIG. 11c), although significant activity can be observed for a range from pH 6.5 to 8.5. The inventors next determined the Kₘ value for the substrate DXP, which is necessary for the calculation of Kᵢ values (inhibition constant) of TgDXR inhibitors. Enzyme activities were measured in the presence of increasing concentrations of DXP (from 10 to 450 μM) and, as shown in FIG. 11d, the Kₘ value of TgDXR for DXP was determined to be 25.5±3.7 μM when fitted into Michaelis-Menten equation. This is comparable to Kₘ values of EcDXR (99 μM) (Deng et al., 2011), PfDXR (106 μM) (Cai et al., 2012) and Mycobacterium tuberculosis DXR (47 μM) (Dhiman et al., 2005).

Upon optimization of the TgDXR enzyme assay conditions, the inhibitory activity of compounds 1-11 (FIG. 2) was determined in order to explore their structure activity relationships (SAR) for this enzyme. These selected compounds represent a broad structural diversity and are particularly suited for the initial SAR study. Fosmidomycin (1) and FR900098 (2) are highly polar phosphonohydroxamic acids, while compounds 3-9 possess more lipophilic properties. Compounds 3 and 4 are phosphonate DXR inhibitors with a pyridine-containing, lipophilic side chain, which was found to be essential for inhibiton (Deng et al., 2011). Hydroxypyridinone compound 5 is the only potent DXR inhibitor without a phosphonate/phosphate group, which also exhibits broad antibacterial activity.¹¹ Pyridine-containing fosmidomycin derivatives 6-9 were recently found to have considerably higher activity against PfDXR as well as the proliferation of P. falciparum (Xue et al., DOG:10.1021/m1300419r), as compared to fosmidomycin. Analogous compounds 10 and 11 possess a 3,4-dichlorophenyl substituent at the a-position, which were also reported to possess potent antimalarial activities (Haemers et al., 2006).

TABLE 2
Kᵢ values of 1-11 against three DXR enzymes.

| Compound | TgDXR (μM) | EcDXR (μM)^{a} | PfDXR (μM)^{a} |
|---|---|---|---|
| 1 | 0.090 | 0.027 | 0.021 |
| 2 | 0.048 | 0.019 | 0.011 |
| 3 | 4.1 | 2.3 | 3.3 |
| 4 | 2.1 | 0.42 | 1.1 |
| 5 | 25.6 | 0.70 | 14.6 |
| 6 | 0.055 | 0.087 | 0.013 |
| 7 | 0.079 | 0.035 | 0.0089 |
| 8 | 0.97 | 0.042 | 0.0019 |
| 9 | 0.53 | 0.082 | 0.013 |
| 10 | 0.077 | 0.058 | 0.015 |
| 11 | 0.22 | 0.036 | 0.025 |

^{a}Data were from (Deng et al., 2011; Xue DOG: 10.1021/ml300419r; Cai et al., 2012).

Table 2 summarizes the Kᵢ values of compounds 1-11 against the DXR enzymes of T. gondii, E. coli and P. falciparum. Fosmidomycin (1) and FR900098 (2) are very strong inhibitors of the T. gondii enzyme with Kᵢ values of 90 and 48 nM. Compounds 3 and 4 without a hydroxamate as metal-binding group are considerably less active, with their Kᵢ values being in the low μM range. The inhibitory activities of the above four compounds against TgDXR are generally in line with those against EcDXR and PfDXR (Table 2). However, the non-phosphonate compound 5 exhibits only very weak inhibitory activity against TgDXR (as observed for PfDXR, the other eukaryotic species) with a Kᵢ value of 25.6 μM. This could explain that despite its high lipophilicity, compound 5 does not block proliferation of T. gondii using our previous method (Nair et al., 2011), although it possesses broad antibacterial activity including E. coli presumably due to its strong activity against EcDXR (Deng et al., 2009). Pyridine-containing compounds 6 and 7 with a formyl group are potent inhibitors of TgDXR with Kᵢ values of 55 and 79 nM, respectively, being more active than their parent compound fosmidomycin. This shows that an appropriate α-substituent may provide favorable interactions with the T. gondii enzyme, as also observed for EcDXR and PfDXR. Surprisingly, the acetyl analogs, compounds 8 and 9, exhibit on average ˜11-fold less activity than compounds 6 and 7, suggesting that with an α-substituent, the terminal methyl group is disfavored on binding to TgDXR. This feature is quite different from those of EcDXR and PfDXR, for which 8 and 9 show similar or even higher activities as compared to their formyl analogs 6 and 7 (Table 2). The same SAR is observed for compounds 10 and 11 against TgDXR, with a formyl group (Kᵢ=77 nM) in 10 showing considerably more inhibitory activity than compound 11 with an acetyl moiety (Kᵢ=220 nM).

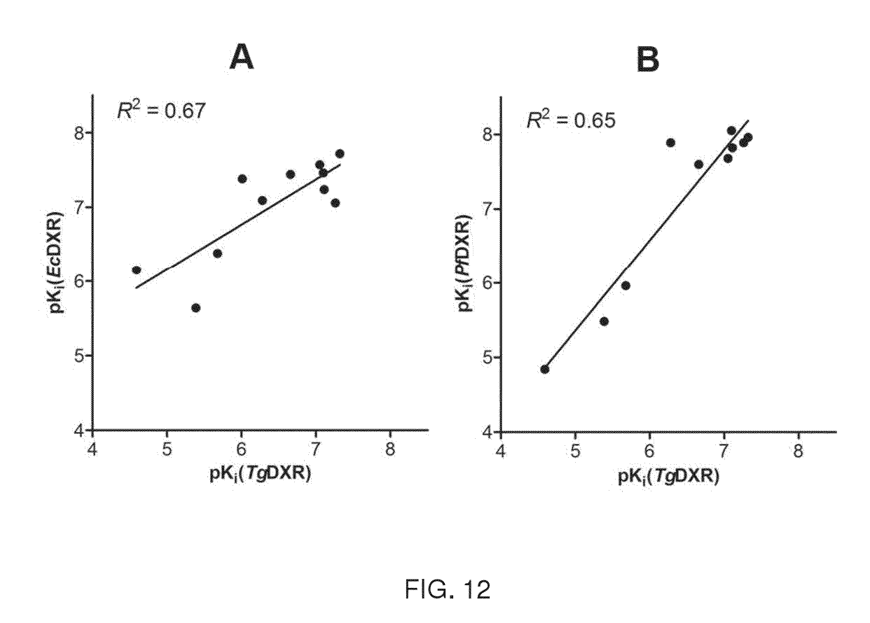

FIG. 12 illustrates the plots of the inhibitory activities of compounds 1-11 against TgDXR with those against EcDXR and PfDXR. Although there are reasonable correlations between the pKiTgDXR and the pKiEcDXR and pKiPfDXR values with R2 of 0.67 and 0.65, respectively, the slope of 0.61 for TgDXR vs. EcDXR is far from the theoretic value of 1 and there are several obvious outliers (out of 11 inhibitors) in these two figures. In addition, the SARs described above also show a different profile for TgDXR inhibition. These comparisons suggest that more biochemical, structural and pharmacological studies of TgDXR are needed to develop effective anti-toxoplasmosis drugs. The methods reported here for expression and inhibition of recombinant TgDXR could therefore be useful for these studies as well as high-throughput screening for potent inhibitors of the enzyme.

Therefore, the inventors therefore expressed and purified recombinant TgDXR, which was found to be enzymatically active. Importantly, the inventors directly support the previous consideration that TgDXR is fully susceptible to fosmidomycin (Nair et al., 2011). TgDXR was observed to exert maximal activity in the presence of 4 mM Mg²⁺ at pH 7.5-8.0. At these conditions, the Kₘ value for the substrate DXP was determined to be 25.5 μM. Thus, a collection of 11 compounds were tested against TgDXR and several potent inhibitors were identified with Kᵢ values as low as 48 nM. Analysis of these results as compared to those of EcDXR and PfDXR revealed a different structure-activity relationship profile for the inhibition of TgDXR.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Bae, Y. A., Cai, G. B., Kim, S. H., Zo, Y. G., and Kong, Y. (2009) Modular evolution of glutathione peroxidase genes in association with different biochemical properties of their encoded proteins in invertebrate animals, BMC evolutionary biology 9, 72.

Bae, Y. A., Kim, S. H., Cai, G. B., Lee, E. G., Kim, T. S., Agatsuma, T., and Kong, Y. (2007) Differential expression of Paragonimus westermani eggshell proteins during the developmental stages, International journal for parasitology 37, 295-305.

Baniecki, M. L., Wirth, D. F., and Clardy, J. (2007) High-throughput Plasmodium falciparum growth assay for malaria drug discovery, Antimicrobial agents and chemotherapy 51, 716-723.

Borrmann, S., Adegnika, A. A., Matsiegui, P. B., Issifou, S., Schindler, A., Mawili-Mboumba, D. P., Baranek, T., Wiesner, J., Jomaa, H., and Kremsner, P. G. (2004) Fosmidomycin-clindamycin for Plasmodium falciparum Infections in African children, The Journal of infectious diseases 189, 901-908.

Borrmann, S., Adegnika, A. A., Moussavou, F., Oyakhirome, S., Esser, G., Matsiegui, P. B., Ramharter, M., Lundgren, I., Kombila, M., Issifou, S., Hutchinson, D., Wiesner, J., Jomaa, H., and Kremsner, P. G. (2005) Short-course regimens of artesunate-fosmidomycin in treatment of uncomplicated Plasmodium falciparum malaria, Antimicrobial agents and chemotherapy 49, 3749-3754.

Borrmann, S., Lundgren, I., Oyakhirome, S., Impouma, B., Matsiegui, P. B., Adegnika, A. A., Issifou, S., Kun, J. F., Hutchinson, D., Wiesner, J., Jomaa, H., and Kremsner, P. G. (2006) Fosmidomycin plus clindamycin for treatment of pediatric patients aged 1 to 14 years with Plasmodium falciparum malaria, Antimicrobial agents and chemotherapy 50, 2713-2718.

Brown, A. C., and Parish, T. (2008) Dxr is essential in Mycobacterium tuberculosis and fosmidomycin resistance is due to a lack of uptake, BMC microbiology 8, 78.

Cai, G. B., Bae, Y. A., Kim, S. H., Na, B. K., Kim, T. S., Jiang, M. S., and Kong, Y. (2006) A membrane-associated metalloprotease of Taenia solium metacestode structurally related to the FACE-1/Ste24p protease family, International journal for parasitology 36, 925-935.

Cai, G. B., Bae, Y. A., Kim, S. H., Sohn, W. M., Lee, Y. S., Jiang, M. S., Kim, T. S., and Kong, Y. (2008) Vitellocyte-specific expression of phospholipid hydroperoxide glutathione peroxidases in Clonorchis sinensis, International journal for parasitology 38, 1613-1623.

Cai, G., Bae, Y., Zhang, Y., He, Y., Jiang, M., and He, L. (2009) Expression and characterization of two tyrosinases from the trematode Schistosoma japonicum, Parasitology research 104, 601-609.

Cai, G., Jiang, M., L., H., Q., Z., Y., Z., X., Y., and X., Z. (2002) Effect of phenol oxidase inhibitor as anti-infective agents against mice infected with Schistosoma japonicum., Chin J Local Dis 21, 267-268.

Cai, G.; Deng, L.; Fryszczyn, B. G.; Brown, N. G.; Liu, Z.; Jiang, H.; Palzkill, T.; Song, Y. ACS Med. Chem. Lett. 2012, 3, 496.

Deng, L., Diao, J., Chen, P., Pujari, V., Yao, Y., Cheng, G., Crick, D. C., Prasad, B. V. V., and Song, Y. (2011) Inhibition of 1-Deoxy-D-Xylulose-5-Phosphate Reductoisomerase by Lipophilic Phosphonates: SAR, QSAR and Crystallographic Studies., Journal of Medicinal Chemistry 54, 4721.

Deng, L., Endo, K., Kato, M., Cheng, G., Yajima, S., and Song, Y. (2011) Structures of 1-Deoxy-D-Xylulose-5 -Phosphate Reductoisomerase/Lipophilic Phosphonate Complexes, ACS Med Chem Lett 2, 165-170.

Deng, L., Sundriyal, S., Rubio, V., Shi, Z., and Song, Y. (2009) A Coordination Chemistry Based Approach to Lipophilic Inhibitors of 1-Deoxy-D-xylulose-5-phosphate Reductoisomerase., Journal of medicinal chemistry 52, 6539-6542.

Devreux, V., Wiesner, J., Jomaa, H., Rozenski, J., Van der Eycken, J., and Van Calenbergh, S. (2007) Divergent strategy for the synthesis of alpha-aryl-substituted fosmidomycin analogues, The Journal of organic chemistry 72, 3783-3789.

Dhiman, R. K., Schaeffer, M. L., Bailey, A. M., Testa, C. A., Scherman, H., and Crick, D. C. (2005) 1-Deoxy-D-xylulose 5-phosphate reductoisomerase (IspC) from Mycobacterium tuberculosis: towards understanding mycobacterial resistance to fosmidomycin, Journal of bacteriology 187, 8395-8402.

Dougherty, D. A. (1996) Cation-pi interactions in chemistry and biology: a new view of benzene, Phe, Tyr, and Trp, Science (New York, N.Y. 271, 163-168.

Gallivan, J. P., and Dougherty, D. A. (1999) Cation-pi interactions in structural biology, Proceedings of the National Academy of Sciences of the United States of America 96, 9459-9464.

Gottlin, E. B., Benson, R. E., Conary, S., Antonio, B., Duke, K., Payne, E. S., Ashraf, S. S., and Christensen, D. J. (2003) High-throughput screen for inhibitors of 1-deoxy-d-xylulose 5-phosphate reductoisomerase by surrogate ligand competition, J Biomol Screen 8, 332-339.

Haemers, T.; Wiesner, J.; Van Poecke, S.; Goeman, J.; Henschker, D.; Beck, E.; Jomaa, H.; Van Calenbergh S. Bioorg. Med. Chem. Lett. 2006, 16, 1888.

He, L., Jiang, M., Cai, G., Yang, M., Yi, X., and Zeng, X. (2001) Effects of acryl thiourea on liver pathologic changes in mice infected with Schistosoma japonicum., Chin J parasitol Parasit Dis 19, 351-353.

Henriksson, L. M., Unge, T., Carlsson, J., Aqvist, J., Mowbray, S. L., and Jones, T. A. (2007) Structures of Mycobacterium tuberculosis 1-deoxy-D-xylulose-5-phosphate reductoisomerase provide new insights into catalysis, The Journal of biological chemistry 282, 19905-19916.

http://www.who.int/en/; Global report on antimalarial efficacy and drug resistance: 2000-2010, in http://www.who.int/malaria/publications/atoz/9789241500470/en/ as of January 2012.

Hunter, W. N. (2007) The non-mevalonate pathway of isoprenoid precursor biosynthesis, The Journal of biological chemistry 282, 21573-21577.

Jomaa, H., Wiesner, J., Sanderbrand, S., Altincicek, B., Weidemeyer, C., Hintz, M., Turbachova, I., Eberl, M., Zeidler, J., Lichtenthaler, H. K., Soldati, D., and Beck, E. (1999) Inhibitors of the nonmevalonate pathway of isoprenoid biosynthesis as antimalarial drugs, Science (New York, N.Y. 285, 1573-1576.

Jones, J. L.; Kruszon-Moran, D.; Sanders-Lewis, K.; Wilson, M. Am. J. Trop. Med. Hyg. 2007, 77, 405.

Kim, H. S., Shibata, Y., Wataya, Y., Tsuchiya, K., Masuyama, A., and Nojima, M. (1999) Synthesis and antimalarial activity of cyclic peroxides, 1,2,4,5,7-pentoxocanes and 1,2,4,5-tetroxanes, Journal of medicinal chemistry 42, 2604-2609.

Kim, S. H., Cai, G. B., Bae, Y. A., Lee, E. G., Lee, Y. S., and Kong, Y. (2009) Two novel phospholipid hydroperoxide glutathione peroxidase genes of Paragonimus westermani induced by oxidative stress, Parasitology 136, 553-565.

Kuntz, L., Tritsch, D., Grosdemange-Billiard, C., Hemmerlin, A., Willem, A., Bach, T. J., and Rohmer, M. (2005) Isoprenoid biosynthesis as a target for antibacterial and antiparasitic drugs: phosphonohydroxamic acids as inhibitors of deoxyxylulose phosphate reducto-isomerase, The Biochemical Journal 386, 127-135.

Kurz, T., Behrendt, C., Pein, M., Kaula, U., Bergmann, B., and Walter, R. D. (2007) gamma-Substituted bis(pivaloyloxymethyl)ester analogues of fosmidomycin and FR900098, Archiv der Pharmazie 340, 661-666.

Kurz, T., Schluter, K., Kaula, U., Bergmann, B., Walter, R. D., and Geffken, D. (2006) Synthesis and antimalarial activity of chain substituted pivaloyloxymethyl ester analogues of Fosmidomycin and FR900098, Bioorganic & medicinal chemistry 14, 5121-5135.

Kurz, T., Schluter, K., Pein, M., Behrendt, C., Bergmann, B., and Walter, R. D. (2007) Conformationally restrained aromatic analogues of fosmidomycin and FR900098, Archiv der Pharmazie 340, 339-344.

Kuzuyama, T., Shimizu, T., Takahashi, S., and Seto, H. (1998) Fosmidomycin, a specific inhibitor of 1-deoxy-xylulose 5-phosphate reductoisomerase in the nonmevalonate pathway for terpenoid biosynthesis, Tetrahedron Letters 39, 7913-7916.

Li, H., Chunsong, H., Cai, G., Qiuping, Z., Qun, L., Xiaolian, Z., Baojun, H., Linjie, Z., Junyan, L., Mingshen, J., and Jinquan, T. (2004) Highly up-regulated CXCR3 expression on eosinophils in mice infected with Schistosoma japonicum, Immunology 111, 107-117.

Mac Sweeney, A., Lange, R., Fernandes, R. P., Schulz, H., Dale, G. E., Douangamath, A., Proteau, P. J., and Oefner, C. (2005) The crystal structure of E. coli 1-deoxy-D-xylulose-5-phosphate reductoisomerase in a ternary complex with the antimalarial compound fosmidomycin and NADPH reveals a tight-binding closed enzyme conformation, Journal of molecular biology 345, 115-127.

Médecins Sans Frontières Access to Essential Medicines Campaign and the Drugs for Neglected Diseases Working Group, S., Fatal Imbalance: The crisis in research and development for drugs for neglected diseases; www.msf.org.

Merckle, L., de Andres-Gomez, A., Dick, B., Cox, R. J., and Godfrey, C. R. (2005) A fragment-based approach to understanding inhibition of 1-deoxy-D-xylulose-5-phosphate reductoisomerase, Chembiochem 6, 1866-1874.

Mine, Y., Kamimura, T., Nonoyama, S., Nishida, M., Goto, S., and Kuwahara, S. (1980) In vitro and in vivo antibacterial activities of FR-31564, a new phosphonic acid antibiotic, The Journal of Antibiotics 33, 36-43.

Missinou, M. A., Borrmann, S., Schindler, A., Issifou, S., Adegnika, A. A., Matsiegui, P. B., Binder, R., Lell, B., Wiesner, J., Baranek, T., Jomaa, H., and Kremsner, P. G. (2002) Fosmidomycin for malaria, Lancet 360, 1941-1942.

Montoya, J. G.; Liesenfeld, O. Lancet 2004, 363, 1965.

Munos, J. W., Pu, X., and Liu, H. W. (2008) Synthesis and analysis of a fluorinated product analogue as an inhibitor for 1-deoxy-D-xylulose 5-phosphate reductoisomerase, Bioorganic & medicinal chemistry letters 18, 3090-3094.

Nair, S. C., Brooks, C. F., Goodman, C. D., Strurm, A., McFadden, G. I., Sundriyal, S., Anglin, J. L., Song, Y., Moreno, S. N., and Striepen, B. (2011) Apicoplast isoprenoid precursor synthesis and the molecular basis of fosmidomycin resistance in Toxoplasma gondii, The Journal of experimental medicine 208, 1547-1559.

Neu, H. C., and Kamimura, T. (1981) In vitro and in vivo antibacterial activity of FR-31564, a phosphonic acid antimicrobial agent, Antimicrobial agents and chemotherapy 19, 1013-1023.

Obiol-Pardo, C., Rubio-Martinez, J. & Imperial, S. Curr. Med. Chem. 2011, 18, 1325.

Ortmann, R., Wiesner, J., Silber, K., Klebe, G., Jomaa, H., and Schlitzer, M. (2007) Novel deoxyxylulosephosphate-reductoisomerase inhibitors: fosmidomycin derivatives with spacious acyl residues, Archiv der Pharmazie 340, 483-490.

Oyakhirome, S., Issifou, S., Pongratz, P., Barondi, F., Ramharter, M., Kun, J. F., Missinou, M. A., Lell, B., and Kremsner, P. G. (2007) Randomized controlled trial of fosmidomycin-clindamycin versus sulfadoxine-pyrimethamine in the treatment of Plasmodium falciparum malaria, Antimicrobial agents and chemotherapy 51, 1869-1871.

Pecoul, B., Chirac, P., Trouiller, P., and Pinel, J. (1999) Access to essential drugs in poor countries: a lost battle?, Jama 281, 361-367.

Ricagno, S., Grolle, S., Bringer-Meyer, S., Sahm, H., Lindqvist, Y., and Schneider, G. (2004) Crystal structure of 1-deoxy-d-xylulose-5-phosphate reductoisomerase from Zymomonas mobilis at 1.9-A resolution, Biochimica et biophysica acta 1698, 37-44.

Rodriguez-Concepcion, M. (2004) The MEP pathway: a new target for the development of herbicides, antibiotics and antimalarial drugs, Current pharmaceutical design 10, 2391-2400.

Sakamoto, Y., Furukawa, S., Ogihara, H., and Yamasaki, M. (2003) Fosmidomycin resistance in adenylate cyclase deficient (cya) mutants of Escherichia coli, Bioscience, biotechnology, and biochemistry 67, 2030-2033.

Shtannikov, A. V., Sergeeva, E. E., Biketov, S. F., and Ostrovskii, D. N. (2007) [Evaluation of in vitro antibacterial activity of fosmidomycin and its derivatives], Antibiotiki i khimioterapiia=Antibiotics and chemoterapy [sic]/Ministerstvo meditsinskoi i mikrobiologicheskoi promyshlennosti SSSR 52, 3-9.

Silber, K., Heidler, P., Kurz, T., and Klebe, G. (2005) AFMoC enhances predictivity of 3D QSAR: a case study with DOXP-reductoisomerase, Journal of medicinal chemistry 48, 3547-3563.

Singh, N., Cheve, G., Avery, M. A., and McCurdy, C. R. (2007) Targeting the methyl erythritol phosphate (MEP) pathway for novel antimalarial, antibacterial and herbicidal drug discovery: inhibition of 1-deoxy-D-xylulose-5-phosphate reductoisomerase (DXR) enzyme, Current pharmaceutical design 13, 1161-1177.

Song, Y., Lin, F. Y., Yin, F., Hensler, M., Rodrigues Poveda, C. A., Mukkamala, D., Cao, R., Wang, H., Morita, C. T., Gonzalez Pacanowska, D., Nizet, V., and Oldfield, E. (2009) Phosphonosulfonates are potent, selective inhibitors of dehydrosqualene synthase and staphyloxanthin biosynthesis in Staphylococcus aureus, Journal of medicinal chemistry 52, 976-988.

Testa, C. A., and Brown, M. J. (2003) The methylerythritol phosphate pathway and its significance as a novel drug target, Current pharmaceutical biotechnology 4, 248-259.

Trouiller, P., Olliaro, P., Torreele, E., Orbinski, J., Laing, R., and Ford, N. (2002) Drug development for neglected diseases: a deficient market and a public-health policy failure, Lancet 359, 2188-2194.

Woo, Y. H., Fernandes, R. P., and Proteau, P. J. (2006) Evaluation of fosmidomycin analogs as inhibitors of the Synechocystis sp. PCC6803 1-deoxy-D-xylulose 5-phosphate reductoisomerase, Bioorganic & medicinal chemistry 14, 2375-2385.

Wu, R., and McMahon, T. B. (2008) Investigation of cation-pi interactions in biological systems, Journal of the American Chemical Society 130, 12554-12555.

Xue, J.; Diao, J.; Cai, G.; Deng, L.; Zheng, B.; Yao, Y.; Song, Y. ACS Med. Chem. Lett, published online ahead of print. DOI: 10.1021/m1300419r.

Yajima, S., Hara, K., Iino, D., Sasaki, Y., Kuzuyama, T., Ohsawa, K., and Seto, H. (2007) Structure of 1-deoxy-D-xylulose 5-phosphate reductoisomerase in a quaternary complex with a magnesium ion, NADPH and the antimalarial drug fosmidomycin, Acta crystallographica 63, 466-470.

Yajima, S., Hara, K., Sanders, J. M., Yin, F., Ohsawa, K., Wiesner, J., Jomaa, H., and Oldfield, E. (2004) Crystallographic structures of two bisphosphonate:1-deoxyxylulose-5-phosphate reductoisomerase complexes, Journal of the American Chemical Society 126, 10824-10825.

Yajima, S., Nonaka, T., Kuzuyama, T., Seto, H., and Ohsawa, K. (2002) Crystal structure of 1-deoxy-D-xylulose 5-phosphate reductoisomerase complexed with cofactors: implications of a flexible loop movement upon substrate binding, Journal of biochemistry 131, 313-317.

Zacharias, N., and Dougherty, D. A. (2002) Cation-pi interactions in ligand recognition and catalysis, Trends in pharmacological sciences 23, 281-287.

Zhang, Y., Cai, G., Jiang, M., He, L., and Yang, M. (2004) Effects of phenol oxidase antigen on liver pathologic changes in mice infected with Schistosoma japonicum., Chin J Schistosoma Control 16, 246-248.

Zhao, Q., He, L., Jiang, M., Cai, G., and Yang, M. (2003) Detection of cytokines of a new mouse model infected with Schistosoma mansoni., Chin J Local Dis 22, 308-309.

One skilled in the art readily appreciates that the present invention is well adapted to carry out the objectives and obtain the ends and advantages mentioned as well as those inherent therein. Methods, procedures, techniques and kits described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. As a composition of matter, a compound of FIG. 3, FIG. 7, FIG. 9, a derivative thereof, or a combination thereof.

2. The composition of claim 1, comprised in a pharmaceutically acceptable carrier.

3. A method of treating and/or preventing a microbial infection in an individual, comprising the step of administering to the individual a therapeutically effective amount of the following:
a) a composition of FIG. 3;
b) a composition of FIG. 7;
c) a composition of FIG. 9;
d) a functionally active derivative of a composition of a), b), or c);
e) a composition comprising at least one phosphate group, a pyridine group, and a hydroxymate; or
f) a mixture thereof;

4. The method of claim 3, wherein the composition is delivered orally, subcutaneously, intramuscularly, topically, rectally, or vaginally.

5. The method of claim 3, wherein the microbial infection is bacterial, viral, fungal, or from a parasitic protist.

6. The method of claim 5, wherein the microbial infection is Plasmodium.

7. The method of claim 6, wherein the microbial infection is Plasmodium falciparum.

8. The method of claim 6, wherein the individual is given an additional treatment for malaria.

9. The method of claim 5, wherein the microbial infection is Toxoplasma gondii.

10. The method of claim 5, wherein the microbial infection is Mycobacterium tuberculosis.

11. A kit comprising the composition of claim 1.
